(19) United States
(12) Patent Application Publication
(10) Pub. No.: US 2016/0060642 A1
(43) Pub. Date: Mar. 3, 2016

(54) G Protein Coupled Receptor Libraries

(71) Applicant: COMMONWEALTH SCIENTIFIC AND INDUSTRIAL RESEARCH ORGANISATION, Campbell, Australian Capital Territory (AU)

(72) Inventors: Stephen Trowell, Oxley (AU); Alisha Anderson, Curtin (AU); Muhammed Tehseen, Queanbeyan (AU); Mira Dumancic, Oxley (AU); Lyndall Briggs, Rivett (AU); Jian Wang, Aranda (AU)

(21) Appl. No.: 14/781,893

(22) PCT Filed: Apr. 16, 2014
(86) PCT No.: PCT/AU2014/000435

§ 371 Date: Oct. 1, 2015

(30) Foreign Application Priority Data
Apr. 16, 2013 (AU) 2013901329
May 30, 2013 (AU) 2013901925

(51) Int. Cl. C12N 15/81; C07K 14/435; G01N 33/74; C12Q 1/02; G01N 33/64

(52) U.S. Cl. CPC C12N 15/81; C12Q 1/025; G01N 33/64; G01N 33/74; C07K 14/43545; G01N 2333/43534; G01N 2333/726

(57) ABSTRACT

The present invention relates to a yeast cell expressing a nematode G protein coupled receptor (GPCR), whereby ligand binding to the GPCR results in a detectable signal. In particular, the present invention relates to a yeast library expressing a range of different GPCRs, and to methods of screening said library.

### FIELD OF THE INVENTION

The present invention relates to a yeast cell expressing a nematode G protein coupled receptor (GPCR), whereby ligand binding to the GPCR results in a detectable signal. In particular, the present invention relates to a yeast library expressing a range of different GPCRs, and to methods of screening said library.

### BACKGROUND OF THE INVENTION

Typically, G protein coupled receptor (GPCR) screening in yeast is performed for a single (or a small number of) GPCR(s) to be identified and validated as a potential target for therapeutic activity or selective toxicity. These are then screened with large and/or diverse chemical libraries in order to identify lead compounds for clinical chemistry and drug development. The screening assay may then be used to aid the process of lead optimisation.

In contrast, the screening of protein libraries expressed in yeast for new protein-ligand interactions relies on a large number of different molecules being reliably expressed in yeast. This leads to challenges in sensitivity and robustness of the assay. This is particularly the case when considering using a relatively uncharacterised class of proteins such as nematode GPCRs, and screening with volatile ligands. These aspects require stable high-level expression of the nematode GPCRs and tightly regulated but strongly ligand inducible expression of the reporter if the screening assay is to have a sufficiently large z factor to be usable.

Minic et al. (2005) relates to a screening assay for mammalian chemoreceptors based on results with the rat olfactory receptor 17 and human OR17-40. Their methodology used an inducible gal promoter to drive expression of the rat OR, rat Gα_{olf} to couple receptor activation to the yeast's endogenous GPCR transduction cascade and the fus-1 promoter to drive expression of the reporter gene firefly luciferase. However, efforts to reproduce this assay, including replacing the mammalian Gα_{olf} with a nematode-yeast chimeric Ga, gpa-1/odr-3, proved unsuccessful (unpublished results).

There is a need for methods of screening yeast libraries expressing nematode GPCRs for the identification of novel receptor-ligand pairs, especially where the ligand is a volatile compound.

### SUMMARY OF THE INVENTION

Nematode receptors comprise a unique GPCR clade (Fredriksson and Schioth, (2005). Various yeast expressing heterologous GPCRs assays have been described, however, the inventors were surprised to find that none worked to any useful extent with nematode GPCRs. Accordingly, the inventors had to develop their own assay.

In a first aspect, the present invention provides a yeast cell comprising

i) a first high copy number extrachromosomal polynucleotide comprising a first polynucleotide encoding a nematode G protein coupled receptor (GPCR) operably linked to a constitutive promoter,

ii) a second high copy number extrachromosomal polynucleotide comprising a second polynucleotide encoding a galactosidase, or a selectable growth marker, operably linked to a promoter activated by the yeast MAP kinase pathway, wherein the promoter is not a FUS-1 promoter,

iii) a mutated yeast gpa-1 gene, and

iv) a third polynucleotide encoding a chimeric G protein comprising the N-terminus of a yeast gpa-1 and at least four C-terminal amino acids of a nematode G protein, operably linked to a promoter,

wherein each promoter directs expression of the polynucleotides in the cell.

In a preferred embodiment, the yeast further has a mutated sst-1 gene and a mutated far-1 gene.

In a further preferred embodiment, the yeast further has a mutated ste-2 gene.

In an embodiment, when a ligand binds the GPCR the resulting z factor is about 0.5 to 1, or about 0.70 to 1, or about 0.8 to 1, or about 0.9 to 1, or between about 0.8 to about 0.98, or between about 0.9 to about 0.98, or about 0.94.

In an embodiment, the promoter activated by the yeast MAP kinase pathway is the FIG-1 or FIG-2 promoter.

In a further embodiment, the constitutive promoter operably linked to the first polynucleotide is selected from the PGK promoter, the ADH-1 promoter, ENO promoter, the PYK-1 promoter, and the CYC-1 promoter.

In yet a further embodiment, the cell comprises at least about 75, or at least about 100, or at least about 150, or between about 75 and about 500, or between about 100 and about 400, or between about 100 and about 250, copies of the first and second high copy number extrachromosomal polynucleotides.

In a preferred embodiment, the yeast is Saccharomyces cerevisiae.

Preferably, the yeast is haploid. In an embodiment, the yeast is mating type a. In instances where the yeast is mating type a it is preferred that the ste-3 gene is mutated rather than the ste-2 gene.

In an embodiment, the third polynucleotide is stably integrated into the genome of the yeast cell.

In an embodiment, the nematode is Caenorhabditis elegans.

In an embodiment, the nematode GPCR is a chemoreceptor, odorant receptor or taste receptor.

As the skilled person would appreciate, a wide variety of different nematode GPCRs can be used in the invention. In an embodiment, the nematode GPCR comprises an amino acid sequence which is at least 80%, at least 90%, at least 95% or at least 99%, identical to one or more of SEQ ID NO's 1 to 297, and/or is encoded by a polynucleotide which comprises a nucleotide sequence which is at least 80%, at least 90%, at least 95% or at least 99%, identical to one or more of SEQ ID NO's 310 to 606.

In an embodiment, the chimeric G protein comprises about 5 C-terminal amino acids of the nematode G protein. In an embodiment, the chimeric G protein comprises an amino acid sequence which is at least 80%, at least 90%, at least 95% or at least 99%, identical to one or more of SEQ ID NO's 298 to 303, and/or is encoded by a 20 polynucleotide which comprises a nucleotide sequence which is at least 80%, at least 90%, at least 95% or at least 99%, identical to one or more of SEQ ID NO's 304 to 309.

In an embodiment, the selectable growth marker is a nutritional marker or antibiotic resistance marker. As the skilled person would appreciate, a wide variety of nutritional markers are useful for the invention including, but not limited to, HIS3, ADE2, URA2, LYS2, ARG2, LEU2, TRP1, MET15, HIS4, URA3, URA5, SFA1, LYS5, ILV2, FBA1, PSE1, PDI1 and PGK1.

In an embodiment, the galactosidase is a β-galactosidase or an α-galactosidase.

In an embodiment, the β-galactosidase is LacZ.

In an embodiment, the promoter operably linked to the third polynucleotide encoding the chimeric G protein is the endogenous gpa-1 promoter.

In an embodiment, the extrachromosomal polynucleotide is a plasmid, cosmid or virus. In a preferred embodiment, the extrachromosomal polynucleotide is a plasmid.

Also provided is a population or library of yeast cells of the invention, wherein at least 10, at least 25, at least 50, at least 100, of the yeast cells have different nematode GPCRs.

In an embodiment, on average each yeast cell comprises at least 100 copies of each of the first and second high copy number extrachromosomal polynucleotide.

In an embodiment, the population or library comprises at least 10, at least 25, at least 50, at least 100, at least 200, at least 250, or all of the nematode GPCRs which comprise an amino acid sequence provided as SEQ ID NO's 1 to 297, or which are at least 80%, at least 90%, at least 95% or at least 99%, identical to one or more of SEQ ID NO's 1 to 297.

Also provided is a composition comprising a yeast cell of the invention, or a population of yeast cells of the invention.

The yeast cells of the invention can be used to identify new ligand/receptor pairs.

Thus, in another aspect, the present invention provides a method of screening for a ligand that binds and activates a nematode G protein coupled receptor, the method comprising

i) contacting a yeast cell of the invention with a candidate ligand, and

ii) determining if the cell expresses galactosidase, or the selectable growth marker,

wherein the presence of galactosidase activity, or activity of the selectable growth marker, indicates that the ligand binds and activates a nematode G protein coupled receptor.

In an embodiment, the z factor of the method when the ligand binds and activates a nematode G protein coupled receptor is about 0.5 to 1, or about 0.70 to 1, or about 0.8 to 1, or about 0.9 to 1, or between about 0.8 to about 0.98, or between about 0.9 to about 0.98, or about 0.94.

In an embodiment, the ligand is volatile.

In a further embodiment, the ligand is from a library of compounds.

In a further embodiment, the method comprises screening a population or library of the invention.

In an embodiment, at least two or three separate yeast colonies/cultures expressing the same G protein coupled receptor are screened for ligand binding.

In a further aspect, the present invention provides a method of detecting a ligand in a sample, the method comprising

i) contacting at least one yeast cell of the invention which comprises a nematode G protein coupled receptor which binds and is activated by the ligand,

ii) determining if the cell expresses galactosidase, or the selectable growth marker,

wherein the presence of galactosidase activity, or activity of the selectable growth marker, indicates that the ligand is present in the sample.

The present inventors have also identified specific polypeptides which bind cyclohexanone, and hence can be used in methods for detecting this compound.

Accordingly, in a further aspect the present invention provides a method of detecting cyclohexanone in a sample, the method comprising

i) contacting the sample with a polypeptide which comprises an animo acid sequence provided as SEQ ID NO:26 or SEQ ID NO:287, or a variant thereof which binds cyclohexanone, and

ii) detecting whether the polypeptide is bound to cyclohexanone.

In an embodiment, the variant comprises an animo acid sequence which is at least 80%, at least 85%, at least 90%, at least 95%, at least 97%, or at least 99%, identical to SEQ ID NO:26 or SEQ ID NO:287, or a cyclohexanone binding fragment thereof.

In an embodiment, the polypeptide is detectably labelled. In a further embodiment, the polypeptide is labelled with a resonance energy transfer (RET) acceptor and donor pair such that when the cyclohexanone binds the labelled polypeptide the spatial location and/or dipole orientation of the donor molecule relative to the acceptor molecule is altered. In yet a further embodiment, the acceptor molecule is a bioluminescent protein. In a further embodiment, the bioluminescent protein is incorporated into the fifth non-transmembrane loop of the polypeptide, and the acceptor molecule is incorporated into the C-terminus of the polypeptide, or b) the acceptor molecule is incorporated into the fifth non-transmembrane loop of the receptor, and the bioluminescent protein is incorporated into the C-terminus.

In a particularly preferred embodiment, the bioluminescent protein is a Renilla luciferase or a biologically active variant (such as RLuc2 or RLuc8) or fragment thereof, the acceptor molecule is green fluorescent protein 2 (GFP²), and the substrate is Coelenterazine 400a.

In an embodiment, the sample is a gas or air sample.

In an embodiment, the cyclohexanone is detected using microfluidics.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Summary of the yeast pheromone responsive pathway, showing key molecules of interest for the development of the screening assay.

FIG. 2. Summary of the yeast GPCR transduction cascade modified to support screening of nematode chemoreceptors with ligands. Compare with FIG. 1.

FIG. 3. Summary of chromosomal and plasmid based genetic modifications required to allow S. cerevisiae to support the screening of nematode chemoreceptors. The Gα-KAGMM chimaera may be replaced with chimaeras between yeast gpa-1 and other nematode Gα proteins.

FIG. 4. Composite fluorescent image showing expression of GFP2 tagged nematode ODR-10 chemoreceptor (OGO) under the control of the constitutive PGK promoter in S. cerevisiae (ste-2, sst-2, far-1 triple mutant) cells. A 20 μl drop of yeast culture was placed on a clean glass slide and stained with Evans Blue dye—a plasma membrane specific dye (0.01% in culture medium, Sigma) for one minute, covered with a cover slip, and observed with a Leica SP2 laser confocal microscope. Samples were excited at 488 nm and images were collected at 510 nm for GFP2 emission and 660 nm for Evans blue emission. Scale bar 10 μm.

FIG. 5. Fluorescence emission scans of Cyb-KAGMM expressing the ODR-10 receptor after exposure to 50 μM or 500 μM diacetyl. A: pGK:Odr10 with Fus1:GFP, B: Biological repeat of “A” under the same conditions, C: pGK:Odr10 with Fig2:GFP. Excitation was at 420 nm. Note that 50 μM diacetyl is below the uninduced condition in panel “C”. VC=vector control.

FIG. 6: Qualitative assay of the reporter gene Lac-Z using 5-bromo-4-chloro-indolyl-β-D-galactopyranoside (X-Gal) as a substrate. Lac-Z expression was driven by the FUS-1 promoter. UN=uninduced.

FIG. 7. Qualitative assay of volatile-induced expression of LacZ using orthonitrophenyl-βD-galactopyranoside (ONPG) as a substrate. The assay was performed using FIG1, FIG2 and FUS1 promoters. The vector control did not include the odr-10 gene. Acetoin was also used as a negative ligand control with the FIG-1 and FIG-2 but not the FUS-1 promoters.

FIG. 8. Quantitative assay of the response of the LacZ reporter gene, driven by the FIG2 promoter, to eleven different concentrations of diacetyl using ONPG as substrate. PGK::Odr-10 (green bars); PGK::H110Y odr-10 mutant (red bars); empty vector control (blue bars). Values are mean and standard deviations of three technical repeats.

FIG. 9. Quantitative assay of the response of the LacZ reporter gene, driven by the FUS1 promoter, to eleven different concentrations of diacetyl using ONPG as substrate. PGK::Odr-10 (green bars); empty vector control (red bars). Values are mean and standard deviations of three technical repeats.

FIG. 10. Molecular phylogeny (sequence relationships) of the 578 members of the str chemoreceptor superfamily of C. elegans. The position of the one odorant receptor previously characterised (odr-10) is shown with an arrow.

FIG. 11. Effects of starting culture dilution and volume of wells on diacetyl-induced lac-Z expression in the lacZ/Odr10/gpa1-KAGMM/ste2⁻ sst2⁻ far1⁻ gpa1⁻ strain of S. cerevisiae following the methods described in Example 2 and throughout the text. Lac-Z expression is estimated by the level of ONPG conversion at Abs_{414 nm} following cell lysis and a 30 minute substrate incubation.

FIG. 12. Plate assay of nematode GPCRs expressed in yeast to and tested (induced) with cyclohexanone. The key for the different wells is provided in Table 2. Colonies B7, D2 and H9 showed significant yellow colour compared to other wells. H9-12 are the control colonies and were tested to 500 μM diacetyl while all the other colonies were tested to 5 mM cyclohexanone.

FIG. 13. Normalized Spectra of plate tested against (induced with) cyclohexanone. Wells B7(), D2 (▪) and positive control H9 (▴) showed a clear peak at 420 nm, indicating they were activated by tested ligands. None of the other wells showed significant peaks at 420 nm, for example, well G6 (*) and the negative control H11 (×).

### KEY TO THE SEQUENCE LISTING

SEQ ID NO: 1 is an amino acid sequence of a Caenorhabditis elegans G protein coupled receptor (GPCR) of Srn-1;

SEQ ID NO: 2 is an amino acid sequence of a C. elegans GPCR of Str-1;

SEQ ID NO: 3 is an amino acid sequence of a C. elegans GPCR of Str-102;

SEQ ID NO: 4 is an amino acid sequence of a C. elegans GPCR of Str-108;

SEQ ID NO: 5 is an amino acid sequence of a C. elegans GPCR of Str-111;

SEQ ID NO: 6 is an amino acid sequence of a C. elegans GPCR of Str-112;

SEQ ID NO: 7 is an amino acid sequence of a C. elegans GPCR of Str-113;

SEQ ID NO: 8 is an amino acid sequence of a C. elegans GPCR of Str-114;

SEQ ID NO: 9 is an amino acid sequence of a C. elegans GPCR of Str-114 SV;

SEQ ID NO: 10 is an amino acid sequence of a C. elegans GPCR of Str-12;

SEQ ID NO: 11 is an amino acid sequence of a C. elegans GPCR of Str-120;

SEQ ID NO: 12 is an amino acid sequence of a C. elegans GPCR of Str-120 SV;

SEQ ID NO: 13 is an amino acid sequence of a C. elegans GPCR of Str-123;

SEQ ID NO: 14 is an amino acid sequence of a C. elegans GPCR of Str-124;

SEQ ID NO: 15 is an amino acid sequence of a C. elegans GPCR of Str-125;

SEQ ID NO: 16 is an amino acid sequence of a C. elegans GPCR of Str-129;

SEQ ID NO: 17 is an amino acid sequence of a C. elegans GPCR of Str-13;

SEQ ID NO: 18 is an amino acid sequence of a C. elegans GPCR of Str-130;

SEQ ID NO: 19 is an amino acid sequence of a C. elegans GPCR of Str-131;

SEQ ID NO: 20 is an amino acid sequence of a C. elegans GPCR of Str-134 V;

SEQ ID NO: 21 is an amino acid sequence of a C. elegans GPCR of Str-135;

SEQ ID NO: 22 is an amino acid sequence of a C. elegans GPCR of Str-139;

SEQ ID NO: 23 is an amino acid sequence of a C. elegans GPCR of Str-14;

SEQ ID NO: 24 is an amino acid sequence of a C. elegans GPCR of Str-141;

SEQ ID NO: 25 is an amino acid sequence of a C. elegans GPCR of Str-143;

SEQ ID NO: 26 is an amino acid sequence of a C. elegans GPCR of Str-144;

SEQ ID NO: 27 is an amino acid sequence of a C. elegans GPCR of Str-146;

SEQ ID NO: 28 is an amino acid sequence of a C. elegans GPCR of Str-148;

SEQ ID NO: 29 is an amino acid sequence of a C. elegans GPCR of Str-151;

SEQ ID NO: 30 is an amino acid sequence of a C. elegans GPCR of Str-153;

SEQ ID NO: 31 is an amino acid sequence of a C. elegans GPCR of Str-155;

SEQ ID NO: 32 is an amino acid sequence of a C. elegans GPCR of Str-159;

SEQ ID NO: 33 is an amino acid sequence of a C. elegans GPCR of Str-162;

SEQ ID NO: 34 is an amino acid sequence of a C. elegans GPCR of Str-163;

SEQ ID NO: 35 is an amino acid sequence of a C. elegans GPCR of Str-164;

SEQ ID NO: 36 is an amino acid sequence of a C. elegans GPCR of Str-165;

SEQ ID NO: 37 is an amino acid sequence of a C. elegans GPCR of Str-166;

SEQ ID NO: 38 is an amino acid sequence of a C. elegans GPCR of Str-168;

SEQ ID NO: 39 is an amino acid sequence of a C. elegans GPCR of Str-169a;

SEQ ID NO: 40 is an amino acid sequence of a C. elegans GPCR of Str-170;

SEQ ID NO: 41 is an amino acid sequence of a C. elegans GPCR of Str-171;

SEQ ID NO: 42 is an amino acid sequence of a C. elegans GPCR of Str-172;

SEQ ID NO: 43 is an amino acid sequence of a C. elegans GPCR of Str-173;

SEQ ID NO: 44 is an amino acid sequence of a C. elegans GPCR of Str-174a;

SEQ ID NO: 45 is an amino acid sequence of a C. elegans GPCR of Str-174 SV;

SEQ ID NO: 46 is an amino acid sequence of a C. elegans GPCR of Str-177;

SEQ ID NO: 47 is an amino acid sequence of a C. elegans GPCR of Str-178;

SEQ ID NO: 48 is an amino acid sequence of a C. elegans GPCR of Str-180a;

SEQ ID NO: 49 is an amino acid sequence of a C. elegans GPCR of Str-180b;

SEQ ID NO: 50 is an amino acid sequence of a C. elegans GPCR of Str-181;

SEQ ID NO: 51 is an amino acid sequence of a C. elegans GPCR of Str-182;

SEQ ID NO: 52 is an amino acid sequence of a C. elegans GPCR of Str-183;

SEQ ID NO: 53 is an amino acid sequence of a C. elegans GPCR of Str-185;

SEQ ID NO: 54 is an amino acid sequence of a C. elegans GPCR of Str-19;

SEQ ID NO: 55 is an amino acid sequence of a C. elegans GPCR of Str-190;

SEQ ID NO: 56 is an amino acid sequence of a C. elegans GPCR of Str-193;

SEQ ID NO: 57 is an amino acid sequence of a C. elegans GPCR of Str-198;

SEQ ID NO: 58 is an amino acid sequence of a C. elegans GPCR of Str-2;

SEQ ID NO: 59 is an amino acid sequence of a C. elegans GPCR of Str-20;

SEQ ID NO: 60 is an amino acid sequence of a C. elegans GPCR of Str-20 SV;

SEQ ID NO: 61 is an amino acid sequence of a C. elegans GPCR of Str-204;

SEQ ID NO: 62 is an amino acid sequence of a C. elegans GPCR of Str-205;

SEQ ID NO: 63 is an amino acid sequence of a C. elegans GPCR of Str-207;

SEQ ID NO: 64 is an amino acid sequence of a C. elegans GPCR of Str-211;

SEQ ID NO: 65 is an amino acid sequence of a C. elegans GPCR of Str-214;

SEQ ID NO: 66 is an amino acid sequence of a C. elegans GPCR of Str-220 SV;

SEQ ID NO: 67 is an amino acid sequence of a C. elegans GPCR of Str-221;

SEQ ID NO: 68 is an amino acid sequence of a C. elegans GPCR of Str-222;

SEQ ID NO: 69 is an amino acid sequence of a C. elegans GPCR of Str-224 SV;

SEQ ID NO: 70 is an amino acid sequence of a C. elegans GPCR of Str-225;

SEQ ID NO: 71 is an amino acid sequence of a C. elegans GPCR of Str-227;

SEQ ID NO: 72 is an amino acid sequence of a C. elegans GPCR of Str-229;

SEQ ID NO: 73 is an amino acid sequence of a C. elegans GPCR of Str-23;

SEQ ID NO: 74 is an amino acid sequence of a C. elegans GPCR of Str-230;

SEQ ID NO: 75 is an amino acid sequence of a C. elegans GPCR of Str-231;

SEQ ID NO: 76 is an amino acid sequence of a C. elegans GPCR of Str-232;

SEQ ID NO: 77 is an amino acid sequence of a C. elegans GPCR of Str-233 SV;

SEQ ID NO: 78 is an amino acid sequence of a C. elegans GPCR of Str-243;

SEQ ID NO: 79 is an amino acid sequence of a C. elegans GPCR of Str-245;

SEQ ID NO: 80 is an amino acid sequence of a C. elegans GPCR of Str-246;

SEQ ID NO: 81 is an amino acid sequence of a C. elegans GPCR of Str-247;

SEQ ID NO: 82 is an amino acid sequence of a C. elegans GPCR of Str-248 SV;

SEQ ID NO: 83 is an amino acid sequence of a C. elegans GPCR of Str-25;

SEQ ID NO: 84 is an amino acid sequence of a C. elegans GPCR of Str-250;

SEQ ID NO: 85 is an amino acid sequence of a C. elegans GPCR of Str-252;

SEQ ID NO: 86 is an amino acid sequence of a C. elegans GPCR of Str-253;

SEQ ID NO: 87 is an amino acid sequence of a C. elegans GPCR of Str-256;

SEQ ID NO: 88 is an amino acid sequence of a C. elegans GPCR of Str-257;

SEQ ID NO: 89 is an amino acid sequence of a C. elegans GPCR of Str-258;

SEQ ID NO: 90 is an amino acid sequence of a C. elegans GPCR of Str-260;

SEQ ID NO: 91 is an amino acid sequence of a C. elegans GPCR of Str-261;

SEQ ID NO: 92 is an amino acid sequence of a C. elegans GPCR of Str-262;

SEQ ID NO: 93 is an amino acid sequence of a C. elegans GPCR of Str-264;

SEQ ID NO: 94 is an amino acid sequence of a C. elegans GPCR of Str-267;

SEQ ID NO: 95 is an amino acid sequence of a C. elegans GPCR of Str-27 SV;

SEQ ID NO: 96 is an amino acid sequence of a C. elegans GPCR of Str-3;

SEQ ID NO: 97 is an amino acid sequence of a C. elegans GPCR of Str-30;

SEQ ID NO: 98 is an amino acid sequence of a C. elegans GPCR of Str-31;

SEQ ID NO: 99 is an amino acid sequence of a C. elegans GPCR of Str-32;

SEQ ID NO: 100 is an amino acid sequence of a C. elegans GPCR of Str-37;

SEQ ID NO: 101 is an amino acid sequence of a C. elegans GPCR of Str-38;

SEQ ID NO: 102 is an amino acid sequence of a C. elegans GPCR of Str-4;

SEQ ID NO: 103 is an amino acid sequence of a C. elegans GPCR of Str-41;

SEQ ID NO: 104 is an amino acid sequence of a C. elegans GPCR of Str-44;

SEQ ID NO: 105 is an amino acid sequence of a C. elegans GPCR of Str-45;

SEQ ID NO: 106 is an amino acid sequence of a C. elegans GPCR of Str-46;

SEQ ID NO: 107 is an amino acid sequence of a C. elegans GPCR of Str-47;

SEQ ID NO: 108 is an amino acid sequence of a C. elegans GPCR of Str-5;

SEQ ID NO: 109 is an amino acid sequence of a C. elegans GPCR of Str-55;

SEQ ID NO: 110 is an amino acid sequence of a C. elegans GPCR of Str-56;

SEQ ID NO: 111 is an amino acid sequence of a C. elegans GPCR of Str-6;

SEQ ID NO: 112 is an amino acid sequence of a C. elegans GPCR of Str-63;

SEQ ID NO: 113 is an amino acid sequence of a C. elegans GPCR of Str-64;

SEQ ID NO: 114 is an amino acid sequence of a C. elegans GPCR of Str-66;

SEQ ID NO: 115 is an amino acid sequence of a C. elegans GPCR of Str-7;

SEQ ID NO: 116 is an amino acid sequence of a C. elegans GPCR of Str-71;

SEQ ID NO: 117 is an amino acid sequence of a C. elegans GPCR of Str-77;

SEQ ID NO: 118 is an amino acid sequence of a C. elegans GPCR of Str-78;

SEQ ID NO: 119 is an amino acid sequence of a C. elegans GPCR of Str-79 SV;

SEQ ID NO: 120 is an amino acid sequence of a C. elegans GPCR of Str-8 SV;

SEQ ID NO: 121 is an amino acid sequence of a C. elegans GPCR of Str-82;

SEQ ID NO: 122 is an amino acid sequence of a C. elegans GPCR of Str-84;

SEQ ID NO: 123 is an amino acid sequence of a C. elegans GPCR of Str-85;

SEQ ID NO: 124 is an amino acid sequence of a C. elegans GPCR of Str-87;

SEQ ID NO: 125 is an amino acid sequence of a C. elegans GPCR of Str-88;

SEQ ID NO: 126 is an amino acid sequence of a C. elegans GPCR of Str-89;

SEQ ID NO: 127 is an amino acid sequence of a C. elegans GPCR of Str-9;

SEQ ID NO: 128 is an amino acid sequence of a C. elegans GPCR of Str-90;

SEQ ID NO: 129 is an amino acid sequence of a C. elegans GPCR of Str-92;

SEQ ID NO: 130 is an amino acid sequence of a C. elegans GPCR of Str-93;

SEQ ID NO: 131 is an amino acid sequence of a C. elegans GPCR of Str-94;

SEQ ID NO: 132 is an amino acid sequence of a C. elegans GPCR of Str-96;

SEQ ID NO: 133 is an amino acid sequence of a C. elegans GPCR of Str-97;

SEQ ID NO: 134 is an amino acid sequence of a C. elegans GPCR of Str-99 SV;

SEQ ID NO: 135 is an amino acid sequence of a C. elegans GPCR of Srh-1;

SEQ ID NO: 136 is an amino acid sequence of a C. elegans GPCR of Srh-10;

SEQ ID NO: 137 is an amino acid sequence of a C. elegans GPCR of Srh-104;

SEQ ID NO: 138 is an amino acid sequence of a C. elegans GPCR of Srh-112;

SEQ ID NO: 139 is an amino acid sequence of a C. elegans GPCR of Srh-115-2;

SEQ ID NO: 140 is an amino acid sequence of a C. elegans GPCR of Srh-118;

SEQ ID NO: 141 is an amino acid sequence of a C. elegans GPCR of Srh-120;

SEQ ID NO: 142 is an amino acid sequence of a C. elegans GPCR of Srh-123;

SEQ ID NO: 143 is an amino acid sequence of a C. elegans GPCR of Srh-128;

SEQ ID NO: 144 is an amino acid sequence of a C. elegans GPCR of Srh-129;

SEQ ID NO: 145 is an amino acid sequence of a C. elegans GPCR of Srh-130;

SEQ ID NO: 146 is an amino acid sequence of a C. elegans GPCR of Srh-132;

SEQ ID NO: 147 is an amino acid sequence of a C. elegans GPCR of Srh-133;

SEQ ID NO: 148 is an amino acid sequence of a C. elegans GPCR of Srh-134;

SEQ ID NO: 149 is an amino acid sequence of a C. elegans GPCR of Srh-138;

SEQ ID NO: 150 is an amino acid sequence of a C. elegans GPCR of Srh-142;

SEQ ID NO: 151 is an amino acid sequence of a C. elegans GPCR of Srh-147;

SEQ ID NO: 152 is an amino acid sequence of a C. elegans GPCR of Srh-149;

SEQ ID NO: 153 is an amino acid sequence of a C. elegans GPCR of Srh-149-2;

SEQ ID NO: 154 is an amino acid sequence of a C. elegans GPCR of Srh-15;

SEQ ID NO: 155 is an amino acid sequence of a C. elegans GPCR of Srh-159;

SEQ ID NO: 156 is an amino acid sequence of a C. elegans GPCR of Srh-166;

SEQ ID NO: 157 is an amino acid sequence of a C. elegans GPCR of Srh-167;

SEQ ID NO: 158 is an amino acid sequence of a C. elegans GPCR of Srh-17;

SEQ ID NO: 159 is an amino acid sequence of a C. elegans GPCR of Srh-174;

SEQ ID NO: 160 is an amino acid sequence of a C. elegans GPCR of Srh-178;

SEQ ID NO: 161 is an amino acid sequence of a C. elegans GPCR of Srh-179;

SEQ ID NO: 162 is an amino acid sequence of a C. elegans GPCR of Srh-18;

SEQ ID NO: 163 is an amino acid sequence of a C. elegans GPCR of Srh-182;

SEQ ID NO: 164 is an amino acid sequence of a C. elegans GPCR of Srh-183;

SEQ ID NO: 165 is an amino acid sequence of a C. elegans GPCR of Srh-184;

SEQ ID NO: 166 is an amino acid sequence of a C. elegans GPCR of Srh-190;

SEQ ID NO: 167 is an amino acid sequence of a C. elegans GPCR of Srh-192;

SEQ ID NO: 168 is an amino acid sequence of a C. elegans GPCR of Srh-193;

SEQ ID NO: 169 is an amino acid sequence of a C. elegans GPCR of Srh-195;

SEQ ID NO: 170 is an amino acid sequence of a C. elegans GPCR of Srh-199;

SEQ ID NO: 171 is an amino acid sequence of a C. elegans GPCR of Srh-2;

SEQ ID NO: 172 is an amino acid sequence of a C. elegans GPCR of Srh-201;

SEQ ID NO: 173 is an amino acid sequence of a C. elegans GPCR of Srh-207;

SEQ ID NO: 174 is an amino acid sequence of a C. elegans GPCR of Srh-208;

SEQ ID NO: 175 is an amino acid sequence of a C. elegans GPCR of Srh-209;

SEQ ID NO: 176 is an amino acid sequence of a C. elegans GPCR of Srh-21;

SEQ ID NO: 177 is an amino acid sequence of a C. elegans GPCR of Srh-211;

SEQ ID NO: 178 is an amino acid sequence of a C. elegans GPCR of Srh-212;

SEQ ID NO: 179 is an amino acid sequence of a C. elegans GPCR of Srh-213;

SEQ ID NO: 180 is an amino acid sequence of a C. elegans GPCR of Srh-214;

SEQ ID NO: 181 is an amino acid sequence of a C. elegans GPCR of Srh-215;

SEQ ID NO: 182 is an amino acid sequence of a C. elegans GPCR of Srh-218;

SEQ ID NO: 183 is an amino acid sequence of a C. elegans GPCR of Srh-233;

SEQ ID NO: 184 is an amino acid sequence of a C. elegans GPCR of Srh-234;

SEQ ID NO: 185 is an amino acid sequence of a C. elegans GPCR of Srh-235;

SEQ ID NO: 186 is an amino acid sequence of a C. elegans GPCR of Srh-239;

SEQ ID NO: 187 is an amino acid sequence of a C. elegans GPCR of Srh-24;

SEQ ID NO: 188 is an amino acid sequence of a C. elegans GPCR of Srh-241;

SEQ ID NO: 189 is an amino acid sequence of a C. elegans GPCR of Srh-255;

SEQ ID NO: 190 is an amino acid sequence of a C. elegans GPCR of Srh-269;

SEQ ID NO: 191 is an amino acid sequence of a C. elegans GPCR of Srh-270;

SEQ ID NO: 192 is an amino acid sequence of a C. elegans GPCR of Srh-271;

SEQ ID NO: 193 is an amino acid sequence of a C. elegans GPCR of Srh-275;

SEQ ID NO: 194 is an amino acid sequence of a C. elegans GPCR of Srh-276;

SEQ ID NO: 195 is an amino acid sequence of a C. elegans GPCR of Srh-277;

SEQ ID NO: 196 is an amino acid sequence of a C. elegans GPCR of Srh-278;

SEQ ID NO: 197 is an amino acid sequence of a C. elegans GPCR of Srh-279;

SEQ ID NO: 198 is an amino acid sequence of a C. elegans GPCR of Srh-281;

SEQ ID NO: 199 is an amino acid sequence of a C. elegans GPCR of Srh-282;

SEQ ID NO: 200 is an amino acid sequence of a C. elegans GPCR of Srh-296;

SEQ ID NO: 201 is an amino acid sequence of a C. elegans GPCR of Srh-30;

SEQ ID NO: 202 is an amino acid sequence of a C. elegans GPCR of Srh-31;

SEQ ID NO: 203 is an amino acid sequence of a C. elegans GPCR of Srh-33;

SEQ ID NO: 204 is an amino acid sequence of a C. elegans GPCR of Srh-37;

SEQ ID NO: 205 is an amino acid sequence of a C. elegans GPCR of Srh-46;

SEQ ID NO: 206 is an amino acid sequence of a C. elegans GPCR of Srh-48;

SEQ ID NO: 207 is an amino acid sequence of a C. elegans GPCR of Srh-50;

SEQ ID NO: 208 is an amino acid sequence of a C. elegans GPCR of Sri-1;

SEQ ID NO: 209 is an amino acid sequence of a C. elegans GPCR of Sri-2;

SEQ ID NO: 210 is an amino acid sequence of a C. elegans GPCR of Sri-7;

SEQ ID NO: 211 is an amino acid sequence of a C. elegans GPCR of Sri-10;

SEQ ID NO: 212 is an amino acid sequence of a C. elegans GPCR of Sri-20;

SEQ ID NO: 213 is an amino acid sequence of a C. elegans GPCR of Sri-21;

SEQ ID NO: 214 is an amino acid sequence of a C. elegans GPCR of Sri-25;

SEQ ID NO: 215 is an amino acid sequence of a C. elegans GPCR of Sri-28;

SEQ ID NO: 216 is an amino acid sequence of a C. elegans GPCR of Sri-29;

SEQ ID NO: 217 is an amino acid sequence of a C. elegans GPCR of Sri-30;

SEQ ID NO: 218 is an amino acid sequence of a C. elegans GPCR of Sri-31;

SEQ ID NO: 219 is an amino acid sequence of a C. elegans GPCR of Sri-38;

SEQ ID NO: 220 is an amino acid sequence of a C. elegans GPCR of Sri-42;

SEQ ID NO: 221 is an amino acid sequence of a C. elegans GPCR of Sri-43;

SEQ ID NO: 222 is an amino acid sequence of a C. elegans GPCR of Sri-46;

SEQ ID NO: 223 is an amino acid sequence of a C. elegans GPCR of Sri-48;

SEQ ID NO: 224 is an amino acid sequence of a C. elegans GPCR of Sri-51;

SEQ ID NO: 225 is an amino acid sequence of a C. elegans GPCR of Sri-54;

SEQ ID NO: 226 is an amino acid sequence of a C. elegans GPCR of Sri-57;

SEQ ID NO: 227 is an amino acid sequence of a C. elegans GPCR of Sri-60;

SEQ ID NO: 228 is an amino acid sequence of a C. elegans GPCR of Sri-63;

SEQ ID NO: 229 is an amino acid sequence of a C. elegans GPCR of Sri-66;

SEQ ID NO: 230 is an amino acid sequence of a C. elegans GPCR of Sri-70;

SEQ ID NO: 231 is an amino acid sequence of a C. elegans GPCR of Sri-73;

SEQ ID NO: 232 is an amino acid sequence of a C. elegans GPCR of Sri-77;

SEQ ID NO: 233 is an amino acid sequence of a C. elegans GPCR of Srd-1;

SEQ ID NO: 234 is an amino acid sequence of a C. elegans GPCR of Srd-3;

SEQ ID NO: 235 is an amino acid sequence of a C. elegans GPCR of Srd-5;

SEQ ID NO: 236 is an amino acid sequence of a C. elegans GPCR of Srd-9;

SEQ ID NO: 237 is an amino acid sequence of a C. elegans GPCR of Srd-10;

SEQ ID NO: 238 is an amino acid sequence of a C. elegans GPCR of Srd-11;

SEQ ID NO: 239 is an amino acid sequence of a C. elegans GPCR of Srd-12;

SEQ ID NO: 240 is an amino acid sequence of a C. elegans GPCR of Srd-13;

SEQ ID NO: 241 is an amino acid sequence of a C. elegans GPCR of Srd-15;

SEQ ID NO: 242 is an amino acid sequence of a C. elegans GPCR of Srd-16;

SEQ ID NO: 243 is an amino acid sequence of a C. elegans GPCR of Srd-17;

SEQ ID NO: 244 is an amino acid sequence of a C. elegans GPCR of Srd-18;

SEQ ID NO: 245 is an amino acid sequence of a C. elegans GPCR of Srd-19;

SEQ ID NO: 246 is an amino acid sequence of a C. elegans GPCR of Srd-20;

SEQ ID NO: 247 is an amino acid sequence of a C. elegans GPCR of Srd-23;

SEQ ID NO: 248 is an amino acid sequence of a C. elegans GPCR of Srd-26;

SEQ ID NO: 249 is an amino acid sequence of a C. elegans GPCR of Srd-27;

SEQ ID NO: 250 is an amino acid sequence of a C. elegans GPCR of Srd-30;

SEQ ID NO: 251 is an amino acid sequence of a C. elegans GPCR of Srd-32;

SEQ ID NO: 252 is an amino acid sequence of a C. elegans GPCR of Srd-33;

SEQ ID NO: 253 is an amino acid sequence of a C. elegans GPCR of Srd-38;

SEQ ID NO: 254 is an amino acid sequence of a C. elegans GPCR of Srd-39 SV;

SEQ ID NO: 255 is an amino acid sequence of a C. elegans GPCR of Srd-40 SV;

SEQ ID NO: 256 is an amino acid sequence of a C. elegans GPCR of Srd-41;

SEQ ID NO: 257 is an amino acid sequence of a C. elegans GPCR of Srd-42;

SEQ ID NO: 258 is an amino acid sequence of a C. elegans GPCR of Srd-43 SV;

SEQ ID NO: 259 is an amino acid sequence of a C. elegans GPCR of Srd-45;

SEQ ID NO: 260 is an amino acid sequence of a C. elegans GPCR of Srd-49;

SEQ ID NO: 261 is an amino acid sequence of a C. elegans GPCR of Srd-50 SV;

SEQ ID NO: 262 is an amino acid sequence of a C. elegans GPCR of Srd-51;

SEQ ID NO: 263 is an amino acid sequence of a C. elegans GPCR of Srd-53;

SEQ ID NO: 264 is an amino acid sequence of a C. elegans GPCR of Srd-55;

SEQ ID NO: 265 is an amino acid sequence of a C. elegans GPCR of Srd-59;

SEQ ID NO: 266 is an amino acid sequence of a C. elegans GPCR of Srd-60;

SEQ ID NO: 267 is an amino acid sequence of a C. elegans GPCR of Srd-61;

SEQ ID NO: 268 is an amino acid sequence of a C. elegans GPCR of Srd-63;

SEQ ID NO: 269 is an amino acid sequence of a C. elegans GPCR of Srd-64;

SEQ ID NO: 270 is an amino acid sequence of a C. elegans GPCR of Srd-65;

SEQ ID NO: 271 is an amino acid sequence of a C. elegans GPCR of Srd-66;

SEQ ID NO: 272 is an amino acid sequence of a C. elegans GPCR of Srd-67;

SEQ ID NO: 273 is an amino acid sequence of a C. elegans GPCR of Srd-71;

SEQ ID NO: 274 is an amino acid sequence of a C. elegans GPCR of Srd-72;

SEQ ID NO: 275 is an amino acid sequence of a C. elegans GPCR of Srd-74;

SEQ ID NO: 276 is an amino acid sequence of a C. elegans GPCR of Srj-1;

SEQ ID NO: 277 is an amino acid sequence of a C. elegans GPCR of Srj-4;

SEQ ID NO: 278 is an amino acid sequence of a C. elegans GPCR of Srj-5;

SEQ ID NO: 279 is an amino acid sequence of a C. elegans GPCR of Srj-6;

SEQ ID NO: 280 is an amino acid sequence of a C. elegans GPCR of Srj-7;

SEQ ID NO: 281 is an amino acid sequence of a C. elegans GPCR of Srj-8;

SEQ ID NO: 282 is an amino acid sequence of a C. elegans GPCR of Srj-9;

SEQ ID NO: 283 is an amino acid sequence of a C. elegans GPCR of Srj-11;

SEQ ID NO: 284 is an amino acid sequence of a C. elegans GPCR of Srj-14;

SEQ ID NO: 285 is an amino acid sequence of a C. elegans GPCR of Srj-15;

SEQ ID NO: 286 is an amino acid sequence of a C. elegans GPCR of Srj-19;

SEQ ID NO: 287 is an amino acid sequence of a C. elegans GPCR of Srj-22;

SEQ ID NO: 288 is an amino acid sequence of a C. elegans GPCR of Srj-26;

SEQ ID NO: 289 is an amino acid sequence of a C. elegans GPCR of Srj-27;

SEQ ID NO: 290 is an amino acid sequence of a C. elegans GPCR of Srj-37;

SEQ ID NO: 291 is an amino acid sequence of a C. elegans GPCR of Srj-39;

SEQ ID NO: 292 is an amino acid sequence of a C. elegans GPCR of Srj-44;

SEQ ID NO: 293 is an amino acid sequence of a C. elegans GPCR of Srm-1;

SEQ ID NO: 294 is an amino acid sequence of a C. elegans GPCR of Srm-2;

SEQ ID NO: 295 is an amino acid sequence of a C. elegans GPCR of Srm-3;

SEQ ID NO: 296 is an amino acid sequence of a C. elegans GPCR of Srm-4;

SEQ ID NO: 297 is an amino acid sequence of a C. elegans GPCR of Srm-5;

SEQ ID NO: 298 is an amino acid sequence of an artificial chimeric G protein of Gpa1/Odr3;

SEQ ID NO: 299 is an amino acid sequence of an artificial chimeric G protein of Gpa1/Gpa3;

SEQ ID NO: 300 is an amino acid sequence of an artificial chimeric G protein of Gpa1/Gpa13;

SEQ ID NO: 301 is an amino acid sequence of an artificial chimeric G protein of Gpa1/Gpa2;

SEQ ID NO: 302 is an amino acid sequence of an artificial chimeric G protein of Gpa1/Gpa5;

SEQ ID NO: 303 is an amino acid sequence of an artificial chimeric G protein of Gpa1/Gpa6;

SEQ ID NO: 304 is a nucleotide sequence of an artificial chimeric G protein of Gpa1/Odr3;

SEQ ID NO: 305 is a nucleotide sequence of an artificial chimeric G protein of Gpa1/Gpa3;

SEQ ID NO: 306 is a nucleotide sequence of an artificial chimeric G protein of Gpa1/Gpa13;

SEQ ID NO: 307 is a nucleotide sequence of an artificial chimeric G protein of Gpa1/Gpa2;

SEQ ID NO: 308 is a nucleotide sequence of an artificial chimeric G protein of Gpa1/Gpa5;

SEQ ID NO: 309 is a nucleotide sequence of an artificial chimeric G protein of Gpa1/Gpa6;

SEQ ID NO: 310 is a nucleotide sequence of a C. elegans GPCR of Srn-1;

SEQ ID NO: 311 is a nucleotide sequence of a C. elegans GPCR of Str-1;

SEQ ID NO: 312 is a nucleotide sequence of a C. elegans GPCR of Str-102;

SEQ ID NO: 313 is a nucleotide sequence of a C. elegans GPCR of Str-108;

SEQ ID NO: 314 is a nucleotide sequence of a C. elegans GPCR of Str-111;

SEQ ID NO: 315 is a nucleotide sequence of a C. elegans GPCR of Str-112;

SEQ ID NO: 316 is a nucleotide sequence of a C. elegans GPCR of Str-113;

SEQ ID NO: 317 is a nucleotide sequence of a C. elegans GPCR of Str-114;

SEQ ID NO: 318 is a nucleotide sequence of a C. elegans GPCR of Str-114 SV;

SEQ ID NO: 319 is a nucleotide sequence of a C. elegans GPCR of Str-12;

SEQ ID NO: 320 is a nucleotide sequence of a C. elegans GPCR of Str-120;

SEQ ID NO: 321 is a nucleotide sequence of a C. elegans GPCR of Str-120 SV;

SEQ ID NO: 322 is a nucleotide sequence of a C. elegans GPCR of Str-123;

SEQ ID NO: 323 is a nucleotide sequence of a C. elegans GPCR of Str-124;

SEQ ID NO: 324 is a nucleotide sequence of a C. elegans GPCR of Str-125;

SEQ ID NO: 325 is a nucleotide sequence of a C. elegans GPCR of Str-129;

SEQ ID NO: 326 is a nucleotide sequence of a C. elegans GPCR of Str-13;

SEQ ID NO: 327 is a nucleotide sequence of a C. elegans GPCR of Str-130;

SEQ ID NO: 328 is a nucleotide sequence of a C. elegans GPCR of Str-131;

SEQ ID NO: 329 is a nucleotide sequence of a C. elegans GPCR of Str-134 V;

SEQ ID NO: 330 is a nucleotide sequence of a C. elegans GPCR of Str-135;

SEQ ID NO: 331 is a nucleotide sequence of a C. elegans GPCR of Str-139;

SEQ ID NO: 332 is a nucleotide sequence of a C. elegans GPCR of Str-14;

SEQ ID NO: 333 is a nucleotide sequence of a C. elegans GPCR of Str-141;

SEQ ID NO: 334 is a nucleotide sequence of a C. elegans GPCR of Str-143;

SEQ ID NO: 335 is a nucleotide sequence of a C. elegans GPCR of Str-144;

SEQ ID NO: 336 is a nucleotide sequence of a C. elegans GPCR of Str-146;

SEQ ID NO: 337 is a nucleotide sequence of a C. elegans GPCR of Str-148;

SEQ ID NO: 338 is a nucleotide sequence of a C. elegans GPCR of Str-151;

SEQ ID NO: 339 is a nucleotide sequence of a C. elegans GPCR of Str-153;

SEQ ID NO: 340 is a nucleotide sequence of a C. elegans GPCR of Str-155;

SEQ ID NO: 341 is a nucleotide sequence of a C. elegans GPCR of Str-159;

SEQ ID NO: 342 is a nucleotide sequence of a C. elegans GPCR of Str-162;

SEQ ID NO: 343 is a nucleotide sequence of a C. elegans GPCR of Str-163;

SEQ ID NO: 344 is a nucleotide sequence of a C. elegans GPCR of Str-164;

SEQ ID NO: 345 is a nucleotide sequence of a C. elegans GPCR of Str-165;

SEQ ID NO: 346 is a nucleotide sequence of a C. elegans GPCR of Str-166;

SEQ ID NO: 347 is a nucleotide sequence of a C. elegans GPCR of Str-168;

SEQ ID NO: 348 is a nucleotide sequence of a C. elegans GPCR of Str-169a;

SEQ ID NO: 349 is a nucleotide sequence of a C. elegans GPCR of Str-170;

SEQ ID NO: 350 is a nucleotide sequence of a C. elegans GPCR of Str-171;

SEQ ID NO: 351 is a nucleotide acid sequence of a C. elegans GPCR of Str-172;

SEQ ID NO: 352 is a nucleotide sequence of a C. elegans GPCR of Str-173;

SEQ ID NO: 353 is a nucleotide sequence of a C. elegans GPCR of Str-174a;

SEQ ID NO: 354 is a nucleotide sequence of a C. elegans GPCR of Str-174 SV;

SEQ ID NO: 355 is a nucleotide sequence of a C. elegans GPCR of Str-177;

SEQ ID NO: 356 is a nucleotide sequence of a C. elegans GPCR of Str-178;

SEQ ID NO: 357 is a nucleotide sequence of a C. elegans GPCR of Str-180a;

SEQ ID NO: 358 is a nucleotide sequence of a C. elegans GPCR of Str-180b;

SEQ ID NO: 359 is a nucleotide sequence of a C. elegans GPCR of Str-181;

SEQ ID NO: 360 is a nucleotide sequence of a C. elegans GPCR of Str-182;

SEQ ID NO: 361 is a nucleotide sequence of a C. elegans GPCR of Str-183;

SEQ ID NO: 362 is a nucleotide sequence of a C. elegans GPCR of Str-185;

SEQ ID NO: 363 is a nucleotide sequence of a C. elegans GPCR of Str-19;

SEQ ID NO: 364 is a nucleotide sequence of a C. elegans GPCR of Str-190;

SEQ ID NO: 365 is a nucleotide sequence of a C. elegans GPCR of Str-193;

SEQ ID NO: 366 is a nucleotide sequence of a C. elegans GPCR of Str-198;

SEQ ID NO: 367 is a nucleotide sequence of a C. elegans GPCR of Str-2;

SEQ ID NO: 368 is a nucleotide sequence of a C. elegans GPCR of Str-20;

SEQ ID NO: 369 is a nucleotide sequence of a C. elegans GPCR of Str-20 SV;

SEQ ID NO: 370 is a nucleotide sequence of a C. elegans GPCR of Str-204;

SEQ ID NO: 371 is a nucleotide sequence of a C. elegans GPCR of Str-205;

SEQ ID NO: 372 is a nucleotide sequence of a C. elegans GPCR of Str-207;

SEQ ID NO: 373 is a nucleotide sequence of a C. elegans GPCR of Str-211;

SEQ ID NO: 374 is a nucleotide sequence of a C. elegans GPCR of Str-214;

SEQ ID NO: 375 is a nucleotide sequence of a C. elegans GPCR of Str-220 SV;

SEQ ID NO: 376 is a nucleotide sequence of a C. elegans GPCR of Str-221;

SEQ ID NO: 377 is a nucleotide sequence of a C. elegans GPCR of Str-222;

SEQ ID NO: 378 is a nucleotide sequence of a C. elegans GPCR of Str-224 SV;

SEQ ID NO: 379 is a nucleotide sequence of a C. elegans GPCR of Str-225;

SEQ ID NO: 380 is a nucleotide sequence of a C. elegans GPCR of Str-227;

SEQ ID NO: 381 is a nucleotide sequence of a C. elegans GPCR of Str-229;

SEQ ID NO: 382 is a nucleotide sequence of a C. elegans GPCR of Str-23;

SEQ ID NO: 383 is a nucleotide sequence of a C. elegans GPCR of Str-230;

SEQ ID NO: 384 is a nucleotide sequence of a C. elegans GPCR of Str-231;

SEQ ID NO: 385 is a nucleotide sequence of a C. elegans GPCR of Str-232;

SEQ ID NO: 386 is a nucleotide sequence of a C. elegans GPCR of Str-233 SV;

SEQ ID NO: 387 is a nucleotide sequence of a C. elegans GPCR of Str-243;

SEQ ID NO: 388 is a nucleotide sequence of a C. elegans GPCR of Str-245;

SEQ ID NO: 389 is a nucleotide sequence of a C. elegans GPCR of Str-246;

SEQ ID NO: 390 is a nucleotide sequence of a C. elegans GPCR of Str-247;

SEQ ID NO: 391 is a nucleotide sequence of a C. elegans GPCR of Str-248 SV;

SEQ ID NO: 392 is a nucleotide sequence of a C. elegans GPCR of Str-25;

SEQ ID NO: 393 is a nucleotide sequence of a C. elegans GPCR of Str-250;

SEQ ID NO: 394 is a nucleotide sequence of a C. elegans GPCR of Str-252;

SEQ ID NO: 395 is a nucleotide sequence of a C. elegans GPCR of Str-253;

SEQ ID NO: 396 is a nucleotide sequence of a C. elegans GPCR of Str-256;

SEQ ID NO: 397 is a nucleotide sequence of a C. elegans GPCR of Str-257;

SEQ ID NO: 398 is a nucleotide sequence of a C. elegans GPCR of Str-258;

SEQ ID NO: 399 is a nucleotide sequence of a C. elegans GPCR of Str-260;

SEQ ID NO: 400 is a nucleotide sequence of a C. elegans GPCR of Str-261;

SEQ ID NO: 401 is a nucleotide sequence of a C. elegans GPCR of Str-262;

SEQ ID NO: 402 is a nucleotide sequence of a C. elegans GPCR of Str-264;

SEQ ID NO: 403 is a nucleotide sequence of a C. elegans GPCR of Str-267;

SEQ ID NO: 404 is a nucleotide sequence of a C. elegans GPCR of Str-27 SV;

SEQ ID NO: 405 is a nucleotide sequence of a C. elegans GPCR of Str-3;

SEQ ID NO: 406 is a nucleotide sequence of a C. elegans GPCR of Str-30;

SEQ ID NO: 407 is a nucleotide sequence of a C. elegans GPCR of Str-31;

SEQ ID NO: 408 is a nucleotide sequence of a C. elegans GPCR of Str-32;

SEQ ID NO: 409 is a nucleotide sequence of a C. elegans GPCR of Str-37;

SEQ ID NO: 410 is a nucleotide sequence of a C. elegans GPCR of Str-38;

SEQ ID NO: 411 is a nucleotide sequence of a C. elegans GPCR of Str-4;

SEQ ID NO: 412 is a nucleotide sequence of a C. elegans GPCR of Str-41;

SEQ ID NO: 413 is a nucleotide sequence of a C. elegans GPCR of Str-44;

SEQ ID NO: 414 is a nucleotide sequence of a C. elegans GPCR of Str-45;

SEQ ID NO: 415 is a nucleotide sequence of a C. elegans GPCR of Str-46;

SEQ ID NO: 416 is a nucleotide sequence of a C. elegans GPCR of Str-47;

SEQ ID NO: 417 is a nucleotide sequence of a C. elegans GPCR of Str-5;

SEQ ID NO: 418 is a nucleotide sequence of a C. elegans GPCR of Str-55;

SEQ ID NO: 419 is a nucleotide sequence of a C. elegans GPCR of Str-56;

SEQ ID NO: 420 is a nucleotide sequence of a C. elegans GPCR of Str-6;

SEQ ID NO: 421 is a nucleotide sequence of a C. elegans GPCR of Str-63;

SEQ ID NO: 422 is a nucleotide sequence of a C. elegans GPCR of Str-64;

SEQ ID NO: 423 is a nucleotide sequence of a C. elegans GPCR of Str-66;

SEQ ID NO: 424 is a nucleotide sequence of a C. elegans GPCR of Str-7;

SEQ ID NO: 425 is a nucleotide sequence of a C. elegans GPCR of Str-71;

SEQ ID NO: 426 is a nucleotide sequence of a C. elegans GPCR of Str-77;

SEQ ID NO: 427 is a nucleotide sequence of a C. elegans GPCR of Str-78;

SEQ ID NO: 428 is a nucleotide sequence of a C. elegans GPCR of Str-79 SV;

SEQ ID NO: 429 is a nucleotide sequence of a C. elegans GPCR of Str-8 SV;

SEQ ID NO: 430 is a nucleotide sequence of a C. elegans GPCR of Str-82;

SEQ ID NO: 431 is a nucleotide sequence of a C. elegans GPCR of Str-84;

SEQ ID NO: 432 is a nucleotide sequence of a C. elegans GPCR of Str-85;

SEQ ID NO: 433 is a nucleotide sequence of a C. elegans GPCR of Str-87;

SEQ ID NO: 434 is a nucleotide sequence of a C. elegans GPCR of Str-88;

SEQ ID NO: 435 is a nucleotide sequence of a C. elegans GPCR of Str-89;

SEQ ID NO: 436 is a nucleotide sequence of a C. elegans GPCR of Str-9;

SEQ ID NO: 437 is a nucleotide sequence of a C. elegans GPCR of Str-90;

SEQ ID NO: 438 is a nucleotide sequence of a C. elegans GPCR of Str-92;

SEQ ID NO: 439 is a nucleotide sequence of a C. elegans GPCR of Str-93;

SEQ ID NO: 440 is a nucleotide sequence of a C. elegans GPCR of Str-94;

SEQ ID NO: 441 is a nucleotide sequence of a C. elegans GPCR of Str-96;

SEQ ID NO: 442 is a nucleotide sequence of a C. elegans GPCR of Str-97;

SEQ ID NO: 443 is a nucleotide sequence of a C. elegans GPCR of Str-99 SV;

SEQ ID NO: 444 is a nucleotide sequence of a C. elegans GPCR of Srh-1;

SEQ ID NO: 445 is a nucleotide sequence of a C. elegans GPCR of Srh-10;

SEQ ID NO: 446 is a nucleotide sequence of a C. elegans GPCR of Srh-104;

SEQ ID NO: 447 is a nucleotide sequence of a C. elegans GPCR of Srh-112;

SEQ ID NO: 448 is a nucleotide sequence of a C. elegans GPCR of Srh-115-2;

SEQ ID NO: 449 is a nucleotide sequence of a C. elegans GPCR of Srh-118;

SEQ ID NO: 450 is a nucleotide sequence of a C. elegans GPCR of Srh-120;

SEQ ID NO: 451 is a nucleotide sequence of a C. elegans GPCR of Srh-123;

SEQ ID NO: 452 is a nucleotide sequence of a C. elegans GPCR of Srh-128;

SEQ ID NO: 453 is a nucleotide sequence of a C. elegans GPCR of Srh-129;

SEQ ID NO: 454 is a nucleotide sequence of a C. elegans GPCR of Srh-130;

SEQ ID NO: 455 is a nucleotide sequence of a C. elegans GPCR of Srh-132;

SEQ ID NO: 456 is a nucleotide sequence of a C. elegans GPCR of Srh-133;

SEQ ID NO: 457 is a nucleotide sequence of a C. elegans GPCR of Srh-134;

SEQ ID NO: 458 is a nucleotide sequence of a C. elegans GPCR of Srh-138;

SEQ ID NO: 459 is a nucleotide sequence of a C. elegans GPCR of Srh-142;

SEQ ID NO: 460 is a nucleotide sequence of a C. elegans GPCR of Srh-147;

SEQ ID NO: 461 is a nucleotide sequence of a C. elegans GPCR of Srh-149;

SEQ ID NO: 462 is a nucleotide sequence of a C. elegans GPCR of Srh-149-2;

SEQ ID NO: 463 is a nucleotide sequence of a C. elegans GPCR of Srh-15;

SEQ ID NO: 464 is a nucleotide sequence of a C. elegans GPCR of Srh-159;

SEQ ID NO: 465 is a nucleotide sequence of a C. elegans GPCR of Srh-166;

SEQ ID NO: 466 is a nucleotide sequence of a C. elegans GPCR of Srh-167;

SEQ ID NO: 467 is a nucleotide sequence of a C. elegans GPCR of Srh-17;

SEQ ID NO: 468 is a nucleotide sequence of a C. elegans GPCR of Srh-174;

SEQ ID NO: 469 is a nucleotide sequence of a C. elegans GPCR of Srh-178;

SEQ ID NO: 470 is a nucleotide sequence of a C. elegans GPCR of Srh-179;

SEQ ID NO: 471 is a nucleotide sequence of a C. elegans GPCR of Srh-18;

SEQ ID NO: 472 is a nucleotide sequence of a C. elegans GPCR of Srh-182;

SEQ ID NO: 473 is a nucleotide sequence of a C. elegans GPCR of Srh-183;

SEQ ID NO: 474 is a nucleotide sequence of a C. elegans GPCR of Srh-184;

SEQ ID NO: 475 is a nucleotide sequence of a C. elegans GPCR of Srh-190;

SEQ ID NO: 476 is a nucleotide sequence of a C. elegans GPCR of Srh-192;

SEQ ID NO: 477 is a nucleotide sequence of a C. elegans GPCR of Srh-193;

SEQ ID NO: 478 is a nucleotide sequence of a C. elegans GPCR of Srh-195;

SEQ ID NO: 479 is a nucleotide sequence of a C. elegans GPCR of Srh-199;

SEQ ID NO: 480 is a nucleotide sequence of a C. elegans GPCR of Srh-2;

SEQ ID NO: 481 is a nucleotide sequence of a C. elegans GPCR of Srh-201;

SEQ ID NO: 482 is a nucleotide sequence of a C. elegans GPCR of Srh-207;

SEQ ID NO: 483 is a nucleotide sequence of a C. elegans GPCR of Srh-208;

SEQ ID NO: 484 is a nucleotide sequence of a C. elegans GPCR of Srh-209;

SEQ ID NO: 485 is a nucleotide sequence of a C. elegans GPCR of Srh-21;

SEQ ID NO: 486 is a nucleotide sequence of a C. elegans GPCR of Srh-211;

SEQ ID NO: 487 is a nucleotide sequence of a C. elegans GPCR of Srh-212;

SEQ ID NO: 488 is a nucleotide sequence of a C. elegans GPCR of Srh-213;

SEQ ID NO: 489 is a nucleotide sequence of a C. elegans GPCR of Srh-214;

SEQ ID NO: 490 is a nucleotide sequence of a C. elegans GPCR of Srh-215;

SEQ ID NO: 491 is a nucleotide sequence of a C. elegans GPCR of Srh-218;

SEQ ID NO: 492 is a nucleotide sequence of a C. elegans GPCR of Srh-233;

SEQ ID NO: 493 is a nucleotide sequence of a C. elegans GPCR of Srh-234;

SEQ ID NO: 494 is a nucleotide sequence of a C. elegans GPCR of Srh-235;

SEQ ID NO: 495 is a nucleotide sequence of a C. elegans GPCR of Srh-239;

SEQ ID NO: 496 is a nucleotide sequence of a C. elegans GPCR of Srh-24;

SEQ ID NO: 497 is a nucleotide sequence of a C. elegans GPCR of Srh-241;

SEQ ID NO: 498 is a nucleotide sequence of a C. elegans GPCR of Srh-255;

SEQ ID NO: 499 is a nucleotide sequence of a C. elegans GPCR of Srh-269;

SEQ ID NO: 500 is a nucleotide sequence of a C. elegans GPCR of Srh-270;

SEQ ID NO: 501 is a nucleotide sequence of a C. elegans GPCR of Srh-271;

SEQ ID NO: 502 is a nucleotide sequence of a C. elegans GPCR of Srh-275;

SEQ ID NO: 503 is a nucleotide sequence of a C. elegans GPCR of Srh-276;

SEQ ID NO: 504 is a nucleotide sequence of a C. elegans GPCR of Srh-277;

SEQ ID NO: 505 is a nucleotide sequence of a C. elegans GPCR of Srh-278;

SEQ ID NO: 506 is a nucleotide sequence of a C. elegans GPCR of Srh-279;

SEQ ID NO: 507 is a nucleotide sequence of a C. elegans GPCR of Srh-281;

SEQ ID NO: 508 is a nucleotide sequence of a C. elegans GPCR of Srh-282;

SEQ ID NO: 509 is a nucleotide sequence of a C. elegans GPCR of Srh-296;

SEQ ID NO: 510 is a nucleotide sequence of a C. elegans GPCR of Srh-30;

SEQ ID NO: 511 is a nucleotide sequence of a C. elegans GPCR of Srh-31;

SEQ ID NO: 512 is a nucleotide sequence of a C. elegans GPCR of Srh-33;

SEQ ID NO: 513 is a nucleotide sequence of a C. elegans GPCR of Srh-37;

SEQ ID NO: 514 is a nucleotide sequence of a C. elegans GPCR of Srh-46;

SEQ ID NO: 515 is a nucleotide sequence of a C. elegans GPCR of Srh-48;

SEQ ID NO: 516 is a nucleotide sequence of a C. elegans GPCR of Srh-50;

SEQ ID NO: 517 is a nucleotide sequence of a C. elegans GPCR of Sri-1;

SEQ ID NO: 518 is a nucleotide sequence of a C. elegans GPCR of Sri-2;

SEQ ID NO: 519 is a nucleotide sequence of a C. elegans GPCR of Sri-7;

SEQ ID NO: 520 is a nucleotide sequence of a C. elegans GPCR of Sri-10;

SEQ ID NO: 521 is a nucleotide sequence of a C. elegans GPCR of Sri-20;

SEQ ID NO: 522 is a nucleotide sequence of a C. elegans GPCR of Sri-21;

SEQ ID NO: 523 is a nucleotide sequence of a C. elegans GPCR of Sri-25;

SEQ ID NO: 524 is a nucleotide sequence of a C. elegans GPCR of Sri-28;

SEQ ID NO: 525 is a nucleotide sequence of a C. elegans GPCR of Sri-29;

SEQ ID NO: 526 is a nucleotide sequence of a C. elegans GPCR of Sri-30;

SEQ ID NO: 527 is a nucleotide sequence of a C. elegans GPCR of Sri-31;

SEQ ID NO: 528 is a nucleotide sequence of a C. elegans GPCR of Sri-38;

SEQ ID NO: 529 is a nucleotide sequence of a C. elegans GPCR of Sri-42;

SEQ ID NO: 530 is a nucleotide sequence of a C. elegans GPCR of Sri-43;

SEQ ID NO: 531 is a nucleotide sequence of a C. elegans GPCR of Sri-46;

SEQ ID NO: 532 is a nucleotide sequence of a C. elegans GPCR of Sri-48;

SEQ ID NO: 533 is a nucleotide sequence of a C. elegans GPCR of Sri-51;

SEQ ID NO: 534 is a nucleotide sequence of a C. elegans GPCR of Sri-54;

SEQ ID NO: 535 is a nucleotide sequence of a C. elegans GPCR of Sri-57;

SEQ ID NO: 536 is a nucleotide sequence of a C. elegans GPCR of Sri-60;

SEQ ID NO: 537 is a nucleotide sequence of a C. elegans GPCR of Sri-63;

SEQ ID NO: 538 is a nucleotide sequence of a C. elegans GPCR of Sri-66;

SEQ ID NO: 539 is a nucleotide sequence of a C. elegans GPCR of Sri-70;

SEQ ID NO: 540 is a nucleotide sequence of a C. elegans GPCR of Sri-73;

SEQ ID NO: 541 is a nucleotide sequence of a C. elegans GPCR of Sri-77;

SEQ ID NO: 542 is a nucleotide sequence of a C. elegans GPCR of Srd-1;

SEQ ID NO: 543 is a nucleotide sequence of a C. elegans GPCR of Srd-3;

SEQ ID NO: 544 is a nucleotide sequence of a C. elegans GPCR of Srd-5;

SEQ ID NO: 545 is a nucleotide sequence of a C. elegans GPCR of Srd-9;

SEQ ID NO: 546 is a nucleotide sequence of a C. elegans GPCR of Srd-10;

SEQ ID NO: 547 is a nucleotide sequence of a C. elegans GPCR of Srd-11;

SEQ ID NO: 548 is a nucleotide sequence of a C. elegans GPCR of Srd-12;

SEQ ID NO: 549 is a nucleotide sequence of a C. elegans GPCR of Srd-13;

SEQ ID NO: 550 is a nucleotide sequence of a C. elegans GPCR of Srd-15;

SEQ ID NO: 551 is a nucleotide sequence of a C. elegans GPCR of Srd-16;

SEQ ID NO: 552 is a nucleotide sequence of a C. elegans GPCR of Srd-17;

SEQ ID NO: 553 is a nucleotide sequence of a C. elegans GPCR of Srd-18;

SEQ ID NO: 554 is a nucleotide sequence of a C. elegans GPCR of Srd-19;

SEQ ID NO: 555 is a nucleotide sequence of a C. elegans GPCR of Srd-20;

SEQ ID NO: 556 is a nucleotide sequence of a C. elegans GPCR of Srd-23;

SEQ ID NO: 557 is a nucleotide sequence of a C. elegans GPCR of Srd-26;

SEQ ID NO: 558 is a nucleotide sequence of a C. elegans GPCR of Srd-27;

SEQ ID NO: 559 is a nucleotide sequence of a C. elegans GPCR of Srd-30;

SEQ ID NO: 560 is a nucleotide sequence of a C. elegans GPCR of Srd-32;

SEQ ID NO: 561 is a nucleotide sequence of a C. elegans GPCR of Srd-33;

SEQ ID NO: 562 is a nucleotide sequence of a C. elegans GPCR of Srd-38;

SEQ ID NO: 563 is a nucleotide sequence of a C. elegans GPCR of Srd-39 SV;

SEQ ID NO: 564 is a nucleotide sequence of a C. elegans GPCR of Srd-40 SV;

SEQ ID NO: 565 is a nucleotide sequence of a C. elegans GPCR of Srd-41;

SEQ ID NO: 566 is a nucleotide sequence of a C. elegans GPCR of Srd-42;

SEQ ID NO: 567 is a nucleotide sequence of a C. elegans GPCR of Srd-43 SV;

SEQ ID NO: 568 is a nucleotide sequence of a C. elegans GPCR of Srd-45;

SEQ ID NO: 569 is a nucleotide sequence of a C. elegans GPCR of Srd-49;

SEQ ID NO: 570 is a nucleotide sequence of a C. elegans GPCR of Srd-50 SV;

SEQ ID NO: 571 is a nucleotide sequence of a C. elegans GPCR of Srd-51;

SEQ ID NO: 572 is a nucleotide sequence of a C. elegans GPCR of Srd-53;

SEQ ID NO: 573 is a nucleotide sequence of a C. elegans GPCR of Srd-55;

SEQ ID NO: 574 is a nucleotide sequence of a C. elegans GPCR of Srd-59;

SEQ ID NO: 575 is a nucleotide sequence of a C. elegans GPCR of Srd-60;

SEQ ID NO: 576 is a nucleotide sequence of a C. elegans GPCR of Srd-61;

SEQ ID NO: 577 is a nucleotide sequence of a C. elegans GPCR of Srd-63;

SEQ ID NO: 578 is a nucleotide sequence of a C. elegans GPCR of Srd-64;

SEQ ID NO: 579 is a nucleotide sequence of a C. elegans GPCR of Srd-65;

SEQ ID NO: 580 is a nucleotide sequence of a C. elegans GPCR of Srd-66;

SEQ ID NO: 581 is a nucleotide sequence of a C. elegans GPCR of Srd-67;

SEQ ID NO: 582 is a nucleotide sequence of a C. elegans GPCR of Srd-71;

SEQ ID NO: 583 is a nucleotide sequence of a C. elegans GPCR of Srd-72;

SEQ ID NO: 584 is a nucleotide sequence of a C. elegans GPCR of Srd-74;

SEQ ID NO: 585 is a nucleotide sequence of a C. elegans GPCR of Srj-1;

SEQ ID NO: 586 is a nucleotide sequence of a C. elegans GPCR of Srj-4;

SEQ ID NO: 587 is a nucleotide sequence of a C. elegans GPCR of Srj-5;

SEQ ID NO: 588 is a nucleotide sequence of a C. elegans GPCR of Srj-6;

SEQ ID NO: 589 is a nucleotide sequence of a C. elegans GPCR of Srj-7;

SEQ ID NO: 590 is a nucleotide sequence of a C. elegans GPCR of Srj-8;

SEQ ID NO: 591 is a nucleotide sequence of a C. elegans GPCR of Srj-9;

SEQ ID NO: 592 is a nucleotide sequence of a C. elegans GPCR of Srj-11;

SEQ ID NO: 593 is a nucleotide sequence of a C. elegans GPCR of Srj-14;

SEQ ID NO: 594 is a nucleotide sequence of a C. elegans GPCR of Srj-15;

SEQ ID NO: 595 is a nucleotide sequence of a C. elegans GPCR of Srj-19;

SEQ ID NO: 596 is a nucleotide sequence of a C. elegans GPCR of Srj-22;

SEQ ID NO: 597 is a nucleotide sequence of a C. elegans GPCR of Srj-26;

SEQ ID NO: 598 is a nucleotide sequence of a C. elegans GPCR of Srj-27;

SEQ ID NO: 599 is a nucleotide sequence of a C. elegans GPCR of Srj-37;

SEQ ID NO: 600 is a nucleotide sequence of a C. elegans GPCR of Srj-39;

SEQ ID NO: 601 is a nucleotide sequence of a C. elegans GPCR of Srj-44;

SEQ ID NO: 602 is a nucleotide sequence of a C. elegans GPCR of Srm-1;

SEQ ID NO: 603 is a nucleotide sequence of a C. elegans GPCR of Srm-2;

SEQ ID NO: 604 is a nucleotide sequence of a C. elegans GPCR of Srm-3;

SEQ ID NO: 605 is a nucleotide sequence of a C. elegans GPCR of Srm-4;

SEQ ID NO: 606 is a nucleotide sequence of a C. elegans GPCR of Srm-5.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, yeast genetics, molecular genetics, immunology, immunohistochemistry, protein chemistry, and biochemistry).

Unless otherwise indicated, the recombinant protein and yeast biology techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “z factor” refers to a measure of the response (sensitivity) of the yeast of the invention to a ligand for a given nematode GPCR (Zhang et al., 1999). Z-factor is defined in terms of four parameters: the means and standard deviations of both the positive (p) and negative (n) controls (μ_{P}, σ_{P}, and μₙ, σₙ). Given these values, the Z-factor is defined as:

$$Z \text{-} factor = 1 - \frac{3 ( {σ}_{p} + {σ}_{n} )}{\vert {μ}_{p} - {μ}_{n} \vert} .$$

Generally, Z-factor is estimated from the sample means and sample standard deviations

$$Estimated Z \text{-} factor = 1 - \frac{3 ( {\hat{σ}}_{p} + {\hat{σ}}_{n} )}{\vert {\hat{μ}}_{p} - {\hat{μ}}_{n} \vert} .$$

The “sample” can be any substance or composition suspected of comprising a ligand to be detected. Examples of samples include air, gas, liquid, biological material and soil. The sample may be obtained directly from the environment or source, or may be at least partially purified by a suitable procedure before a method of the invention is performed. For example, the methods of the invention can be used to identify and isolate receptors that respond to chemicals that are relevant in an agricultural production setting, particularly those present at nanomolar or parts per billion ((v/v; w/v; or by molar ratio) levels or lower. Such chemicals include volatile or non-volatile indicators of pathogen presence absence, pest or parasite presence or absence, crop or animal vigour, ripeness or other agronomically useful attribures. In the human health field, the methods of the invention can be used to identify and isolate receptors that selectively respond to volatile or non-volatile markers of infections (e.g. malaria, tuberculosis, influenza, leishmaniasis, bacteraemia, soft tissue infections, urinary tract infections, lungh infections etc.) or non-infectious diseases (e.g. lung cancers, pancreatic cancers, ovarian and other cancers and non-infectious conditions and also genetic deficiency conditions, degenerative diseases and metabolic conditions such as Type I or Type II diabetes, metabolic syndrome etc. or the quality of treatment or control of any infectious or non-infectious health condition). In a food safety and quality context the methods of the invention can be used to identify and isolate receptors that respond to volatile or non-volatile chemicals indicative of the presence of microbial contamination, mycotoxin or other toxin contamination, or the presence of inadvertent contaminants, or deliberate adulterants in food. The methods of the invention can be used to identify and isolate receptors for compounds that indicate particular desirable or undesirable organoleptic attributes, such as ripeness or maturity of fruits, cheeses and other products or contaminants and taints such as cork taint in wine, boar taint in pork, “Bret” taint in wines and other alcoholic beverages or sulphuraceous or other taints in milk and dairy products. The methods of the invention can be used for similar purposes relevant to a wide range of other economic or social activities, including security screening, process control, environmental monitoring, indoor climate control, consumer product development and manufacturing etc. In addition, the identified and isolated receptors can be used in other methods of the invention to detect a compound (ligand) mentioned above.

The “ligand” can be any compound which binds and activates a particular G protein coupled receptor, such as, but not limited to, a peptide, a protein, a hormone, a lipid, a small carbon based molecule, a carbohydrate, an oil, an odorant, a polymer etc.

As used herein, resonance energy transfer (RET) is a proximity assay based on the non-radioactive transfer of energy between a donor molecule and an acceptor molecule.

As used herein, the term “acceptor molecule” refers to any compound which can accept energy emitted as a result of the activity of a donor, and re-emit it as light energy.

As used herein, the term “spatial location” refers to the three dimensional positioning of the donor molecule relative to the acceptor molecule which changes as a result of a compound binding a polypeptide defined herein.

As used herein, the term “dipole orientation” refers to the direction in three-dimensional space of the dipole moment associated either with the donor molecule and/or the acceptor molecule relative their orientation in three-dimensional space. The dipole moment is a consequence of a variation in electrical charge over a molecule.

As used herein, the term “bioluminescent protein” refers to any protein capable of acting on a suitable substrate to generate luminescence.

As used herein, the term “substrate” refers to any molecule that can be used in conjunction with a bioluminescent protein to generate or absorb luminescence.

### Nematode G Protein Coupled Receptors

As used herein, unless specified otherwise, the term “G protein coupled receptor” refers to a seven transmembrane receptor which signals through G proteins. The receptor may be a single subunit, or two or more receptor subunits. When two or more receptor subunits are present they may be the same, different, or a combination thereof (for example, two of one subunit and a single of another subunit). In one embodiment, the yeast cells of the invention comprise a homodimer of the same GPCR subunit. In another embodiment, the yeast cells of the invention comprise a heterodimer of different GPCR subunits. When expressed in a yeast cell, the GPCRs are located in the yeast cell membrane. In addition, when expressed in a yeast cell the GPCR is functionally coupled with an intracellular signalling pathway such as the pheromone response pathway. Furthermore, unless specified or implied otherwise the terms “G protein coupled receptor” and “subunit of a G protein coupled receptor”, or variations thereof, are used interchangeably.

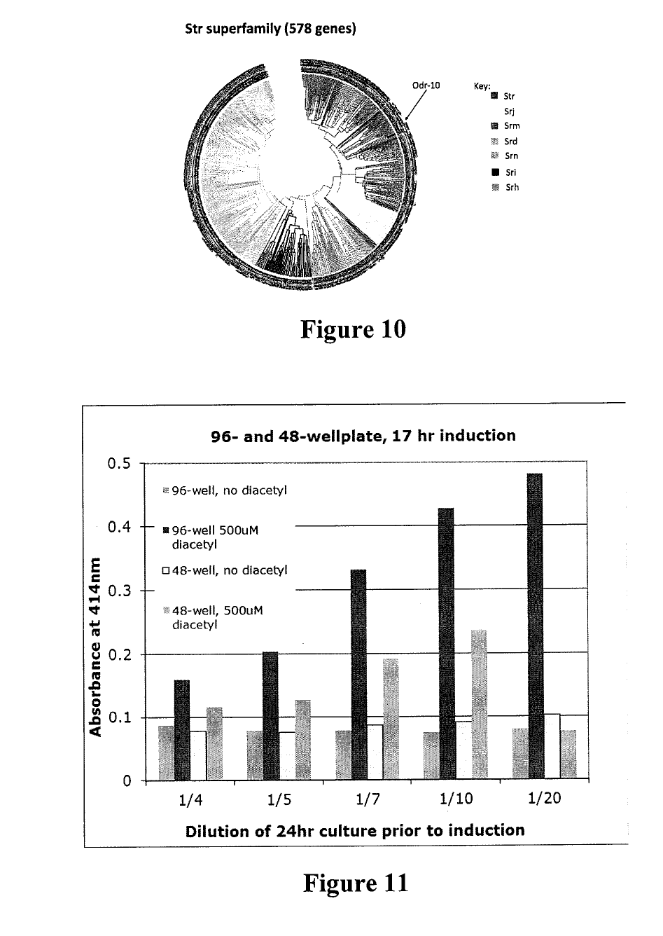

Nematode receptors comprise a unique GPCR clade (Fredriksson and Schioth, 2005) (FIG. 10). The GPCRs useful for the invention may be obtained from a variety of nematodes including, but not limited to, those of the Genera: Caenorhabditis such as C. elegans, Ancylostoma such as Ancylostoma caninum, Anguina, Ditylenchus, Tylenchorhynchus, Pratylenchus, Radopholus, Hirschmanniella, Nacobbus, Hoplolaimus, Scutellonema, Rotylenchus, Helicotylenchus, Rotylenchulus, Belonolaimus, Heterodera such as Heterodera glycines, Meloidogyne such as Meloidogyne javanica, Meloidogyne incognita, and Meloidogyne arenaria, Criconemoides, Hemicycliophora, Paratylenchus, Tylenchulus, Aphelenchoides, Bursaphelenchus, Rhadinaphelenchus, Longidorus, Xiphinema, Trichodorus, Paratrichodorus, Dirofiliaria such as Dirofilaria immitis, Dirofilaria tenuis, Dirofilaria repens, and Dirofilari ursi, Onchocerca, Brugia, Acanthocheilonema, Aelurostrongylus, Ancylostoma, Angiostrongylus, Ascaris, Bunostomum, Capillaria, Chabertia, Cooperia, Crenosoma, Dictyocaulus, Dioctophyme, Dipetalonema, Dracunculus, Enterobius, Filaroides, Haemonchus such as Haemonchus contortus, Lagochilascaris, Loa, Manseonella, Muellerius, Necator, Nematodirus, Oesophagostomum, Ostertagia, Parafilaria, Parascaris, Physaloptera, Protostrongylus, Setaria, Spirocerca, Stephanogilaria, Strongyloides, Strongylus, Thelazia, Toxascaris, Toxocara such as Toxocara canis and Toxocara cati, Trichinella such as Trichinella spiralis and Trichurs muris, Trichostrongylus, Trichuris, Uncinaria, and Wuchereria.

In an embodiment, the GPCR is a chemoreceptor (or putative chemoreceptors), odorant receptor (or putative odorant receptors) or taste receptor (or putative taste receptor) as identified by Robertson et al. (1998 and 2001).

The GPCR may have an amino acid sequence which is naturally occurring or which is a mutant/variant thereof, a biologically active fragment thereof, or a fusion thereof. Amino acid sequence mutants/variants of naturally occurring G protein coupled receptors can be prepared by introducing appropriate nucleotide changes into the encoding polynucleotide, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (variant) polypeptides can be prepared using any technique known in the art. For example, a polynucleotide described herein can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides encoding G protein coupled receptors are subjected to DNA shuffling techniques as broadly described by Harayama (1998). Products derived from mutated/variant DNA can readily be screened using techniques described herein to determine if they are useful for the methods of the invention.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the G protein coupled receptor removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Also included within the scope of the invention are polypeptides which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Furthermore, the GPCR may be a non-naturally occurring chimera of two or more different GPCRs. In particular, this enables a transduction cassette to be produced where portions of one receptor are always present in the chimera into which other portions of a wide variety of GPCRs are inserted depending on the compound to be detected.

In one embodiment, the subunit comprises the N-terminus and at least a majority of the first transmembrane domain of a first G protein coupled receptor subunit, at least a majority of the first non-transmembrane loop through to at least a majority of the fifth transmembrane domain of a second G protein coupled receptor subunit, and at least a majority of the fifth non-transmembrane loop through to the C-terminal end of the first G protein coupled receptor subunit.

In another embodiment, the subunit comprises the N-terminus through to at least a majority of the fifth transmembrane domain of a first G protein coupled receptor subunit, and at least a majority of the fifth non-transmembrane loop through to the C-terminal end of a second G protein coupled receptor subunit.

The skilled person can readily determine the N-terminal end, transmembrane domains, non-transmembrane loops (domains) and C-terminus of a G protein coupled receptor. For example, a variety of bioinformatics approaches may be used to determine the location and topology of transmembrane domains in a protein, based on its amino acid sequence and similarity with known transmembrane domain of G protein coupled receptors. Alignments and amino acid sequence comparisons are routinely performed in the art, for example, by using the BLAST program or the CLUSTAL W program. Based on alignments with known transmembrane domain-containing proteins, it is possible for one skilled in the art to predict the location of transmembrane domains. Furthermore, the 3 dimensional structures of some membrane-spanning proteins are known, for example, the seven transmembrane G-protein coupled rhodopsin photoreceptor structure has been solved by x-ray crystallography. Based on analyses and comparisons with such 3D structures, it may be possible to predict the location and topology of transmembrane domains in other membrane proteins. There are also many programs available for predicting the location and topology of transmembrane domains in proteins. For example, one may use one or a combination of the TMpred (Hofmann and Stoffel, 1993), which predicts membrane spanning proteins segments; TopPred (von Heijne et al., 1992) which predicts the topology of membrane proteins; PREDATOR (Frishman and Argos, 1997), which predicts secondary structure from single and multiple sequences; TMAP (Persson and Argos, 1994), which predicts transmembrane regions of proteins from multiply aligned sequences; and ALOM2 (Klein et al., 1984), which predicts transmembrane regions from single sequences.

In an embodiment, the nematode GPCR comprises a sequence which is at least 40% identical to one or more of SEQ ID NO's 1 to 297.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 50%, more preferably at least 60%, more preferably at least 70%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length. If the GPCR is a chimera of two or more different nematode GPCRs, it is preferred that the alignments are performed independently for each different section of the GPCR from a different molecule.

As used herein, a “biologically active fragment” is a portion of a polypeptide as described herein which maintains a defined activity of the full-length polypeptide. For example, a biologically active fragment of a G protein coupled receptor must be capable of binding the target ligand resulting in activation of a signalling pathway in the yeast cell linked to the reporter gene. Biologically active fragments can be any size as long as they maintain the defined activity. Preferably, biologically active fragments are at least 150, more preferably at least 250 amino acids in length.

As used herein, a “biologically active variant” is a molecule which differs from a naturally occurring and/or defined molecule by one or more amino acids but maintains a defined activity, such as defined above for biologically active fragments. Biologically active variants are typically at least 50%, more preferably at least 80%, more preferably at least 90%, more preferably at least 95%, more preferably at least 97%, and even more preferably at least 99% identical to the naturally occurring and/or defined molecule.

Nematode G protein coupled receptor used in the invention require a suitable signal sequence, such as a hydrophobic N-terminal signal sequence, to direct the receptor after translation to the cell membrane. Such signal sequences are well known in the art. In an embodiment, the native signal sequence of the nematode G protein coupled receptor is used. In an alternate embodiment, the nematode G protein coupled receptor comprises a yeast N-terminal signal sequence which may be cleaved during transport of the receptor to the cell membrane. Examples of such yeast signal sequences include, but are not limited to, the yeast a-mating factor signal sequence or the yeast invertase signal sequence.

### G Proteins

G-proteins are a family of proteins involved in second messenger cascades for intracellular signaling. G proteins function as “molecular switches,” alternating between an inactive guanosine diphosphate (GDP) bound state and an active guanosine triphosphate (GTP) bound state. Ultimately, G proteins regulate downstream cell processes by initiating cascades of signal transduction networks (Hofmann et al., 2009; Oldham and Hamm, 2008).

There are two distinct families of G proteins: Heterotrimeric G proteins, sometimes referred to as the “large” G proteins, that are activated by G protein coupled receptors and made up of alpha (α), beta (β), and gamma (γ) subunits; and “small” G proteins (20-25 kDa) that belong to the Ras superfamily of small GTPases. These proteins are homologous to the alpha (α) subunit found in heterotrimeric G proteins, and also bind GTP and GDP and are involved in signal transduction. In order to associate with the plasma membrane, many G proteins are covalently modified with lipids, for example heterotrimeric G protein subunits may be myristolated, palmitoylated, or prenylated, while small G proteins may be prenylated.

As the skilled addressee will be aware, there are many known nematode G-proteins, as well as routine methods for identifying yet unknown nematode G-proteins. For example, C. elegans has 21 Gα, 2 Gβ and 2 Gγ genes (Jansen et al., 1999; Cuppen et al., 2003). C. elegans expresses one ortholog of each of the mammalian families: GSA-1 (Gs), GOA-1 (Gi/o), EGL-30 (Gq) and GPA-12 (G12). The remaining C. elegans α subunits (GPA-1-11, GPA-13-17 and ODR-3) do not share sufficient homology to allow classification. The conserved Gα subunits, with the exception of GPA-12, are expressed broadly while 14 of the new Gα genes are expressed in subsets of chemosensory neurons.

The Gβ subunit, GPB-1, as well as the Gγ subunit, GPC-2, appear to function along with the α subunits in the classic G protein heterotrimer. The remaining Gβ subunit, GPB-2, is thought to regulate the function of certain RGS proteins, while the remaining Gγ subunit, GPC-1, has a restricted role in chemosensation. The functional difference for most G protein pathways in C. elegans, therefore, resides in the α subunit.

The G protein may have an amino acid sequence which is naturally occurring or which is a mutant/variant thereof, or a biologically active fragment thereof, a fusion thereof. These concepts are well known in the art and described above in relation to the GPCRs. In a preferred embodiment, as part of the signalling cascade the Ga protein dissociates from yeast Gβγ protein, which leads to activation of the MAP kinase signalling pathway and results in reporter gene expression.

The invention, in part, relies on the expression in yeast of a chimeric G protein comprising the N-terminus of a yeast gpa-1 and at least four C-terminal amino acids of a nematode G protein. The production of such chimeric proteins and their use detecting signalling through heterologously expressed GPCRs has been previously described by, for example, Brown et al. (2000), WO 99/14344 and WO 99/18211. In an embodiment, the chimeric G protein comprises about 5 of the C-terminal amino acids of the nematode G protein at the C-terminus of the chimera. In another embodiment, the chimeric G protein comprises about 5 to about 11 of the C-terminal amino acids of the nematode G protein at the C-terminus of the chimera.

In an embodiment, the nematode G protein is gpa-1, gpa-2, gpa-3, gpa-6, gpa-13 or gpa-15.

### Recombinant Yeast

The present invention relates to recombinant yeast cells, and the use thereof to identify ligand binding to a heterologously expressed GPCR.

As used herein, “activates” or variations thereof in relation to the ligand binding a GPCR means that upon binding of the ligand to the receptor an intracellular signaling cascade involving G proteins and the MAP kinase pathway in the yeast is turned on ultimately resulting in expression of the reporter gene.

The yeast cell can be any cell suitable for the invention. In a particularly 10 preferred embodiment, the yeast cell, in a native unmodified state, has a functional G protein coupled receptor signalling pathway such as the pheromone responsive pathway. The yeast cell may be a member of the Saccharomyces genus such as Saccharomyces cerevisiae, Saccharomyces uvae or Saccharomyces kluyveri, Ustilaqo genus such Ustilaqo maydis, Kluyveromyces genus such as Kluyveromyces lactis or Kluyveromyces drosophilarum, Pichia genus such as Pichia pastoris and Pichia membranaefaciens, Schizosaccharomyces genus such as Schizosaccharomyces pombe, Yarrowia genus such as Yarrowia lipolytica, Candida genus such as Candida utilis or Candida cacaoi or Zygosaccharomyces genus such as Zygosaccharomyces rouxii, Zygosaccharomyces bailii and Zygosaccharomyces fermentati.

In a preferred embodiment, the yeast cell is Saccharomyces sp., more preferably Saccharomyces cerevisiae.

In a preferred embodiment, the yeast cell is haploid. In a further preferred embodiment, the yeast cell is mating type a or α.

Yeast cells of the invention have a mutated gpa-1 gene. The term “mutated” when referring to an endogenous yeast gene, such as gpa-1, far-1, sst-1 and ste-2 gene means that expression has been reduced, and/or the function of the encoded normal (wild-type) protein has been diminished (preferably abolished), when compared to a yeast cell with a corresponding normal (wild type) gene. Preferably, no functional protein normally encoded by the gene can be found in the yeast cell. Such mutated genes can be produced by a wide variety of routine procedures such as mutagenesis or gene knockouts. In an embodiment, one, more or all of the far-1, sst-1 and ste-2 genes have been deleted.

Examples of mutated yeast gpa-1 genes are described in Iguchi et al. (2010), Dowell and Brown (2009), Brown et al. (2000), Fukutani et al. (2012), WO 99/14344 and WO 99/18211. Examples of far-1, sst-2 and ste-2 mutated genes are described in Fukutani et al. (2012).

As used herein, “operably linked” refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

“Constitutive promoter” refers to a promoter that directs expression of an operably linked transcribed sequence in the yeast without the need to be induced by specific growth conditions. Examples of constitutive promoters useful for the invention, for instance operably linked to the first polynucleotide, include, but are not limited to, a yeast PGK (phosphoglycerate kinase) promoter, a yeast ADH-1 (alcohol dehydrogenase) promoter, a yeast ENO (enolase) promoter, a yeast glyceraldehyde 3-phosphate dehydrogenase promoter (GPD) promoter, a yeast PYK-1 (pyruvate kinase) promoter, a yeast translation-elongation factor-1-alpha promoter (TEF) promoter and a yeast CYC-1 (cytochrome c-oxidase promoter) promoter. In a preferred embodiment, a yeast promoter is a S. cerevisiae promoter. In another embodiment, the constitutive promoter may not have been derived from yeast. Examples of such promoters useful for the invention include, but are not limited to, the cauliflower mosaic virus 35S promoter, the glucocorticoid response element, and the androgen response element. The constitutive promoter may be the naturally occurring molecule or a variant thereof comprising, for example, one, two or three nucleotide substitutions which do not abolish (and preferably enhance) promoter function.

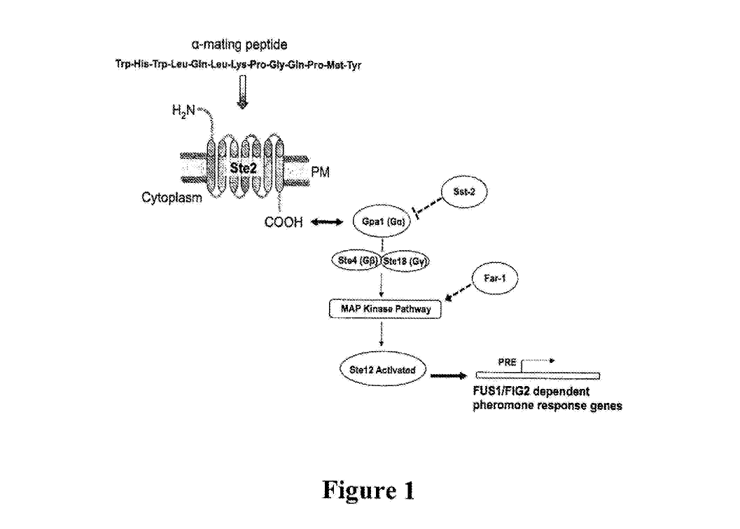

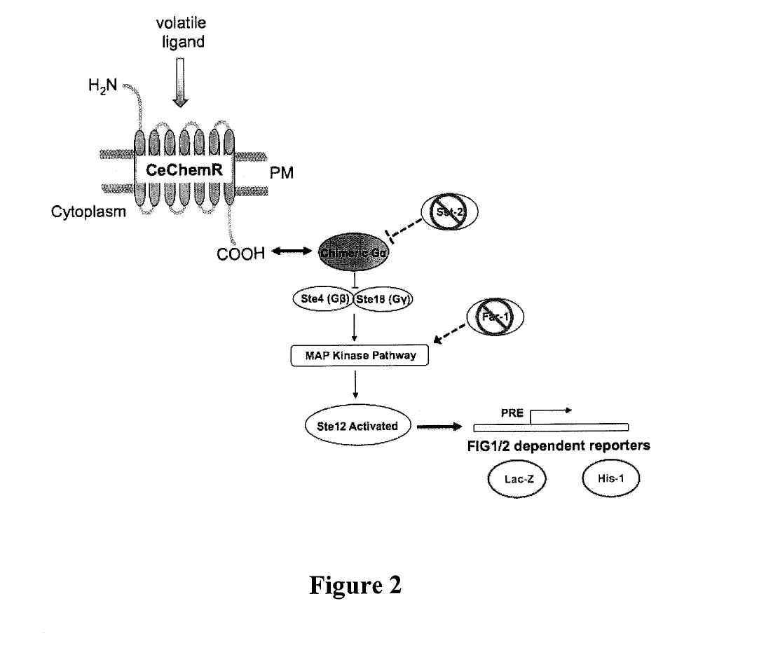

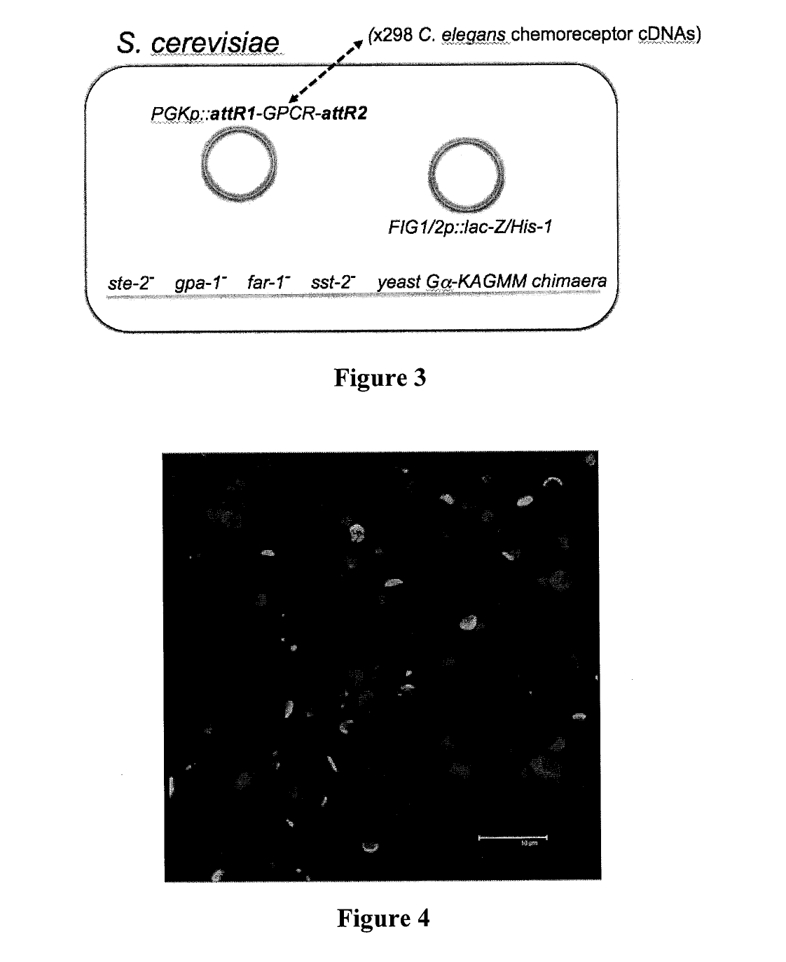

As used herein, the term “promoter activated by the yeast MAP kinase pathway” refers to a promoter which drives gene transcription of an operably linked polynucleotide (nucleic acid) in response to the yeast MAP kinase pathway being activated through binding of a ligand to the GPCR (see FIGS. 1 and 2). Examples of promoter activated by the yeast MAP kinase pathway used in the invention include, but are not limited to, FIG2 (yeast protein involved in mating induction), FIG1 (yeast protein required for efficient mating), FIG4 (yeast Dac1p homolog), PRM1 (yeast pheromone-regulated membrane protein), ERG24, (yeast C-14 sterol reductase), FUS3, (yeast MAPK mediating mating pheromone signalling), PEP1 (yeast receptor for vacuole sorting), YOR129C, HYM1, FAR1 (yeast protein involved in cell cycle arrest) and PCL2 (yeast cyclin protein). In an embodiment, the promoter is bound, and transcription is activated by the yeast Ste12 transcription factor. As mentioned above, the promoter activated by the yeast MAP kinase pathway is not the FUS-1 promoter. The promoter activated by the yeast MAP kinase pathway may be the naturally occurring molecule or a variant thereof comprising, for example, one, two or three nucleotide substitutions which do not abolish (and preferably enhance) promoter function.

The promoter operably linked to the third polynucleotide encoding the chimeric G protein can be any suitable promoter such as a constitutive promoter as described above. Conveniently, in an embodiment, the promoter is the endogenous gpa-1 promoter. In this regard, the gene encoding the chimeric protein is the endogenous gene which has been mutated to express the C-terminal amino acids of a nematode G protein.

As used herein, the term “high copy number” means that the yeast cell comprises at least about 75, or at least about 100, or at least about 150, or between about 75 and about 500, or between about 100 and about 400, or between about 100 and about 250, copies of the extrachromosomal polynucleotide. In an embodiment, the extrachromosomal polynucleotide is a plasmid, cosmid or virus, preferably a plasmid. Alternatively, when referring to a population (and/or library) of yeast cells of the invention, the term “high copy number” means that on average each yeast cell comprises at least about 75, or at least about 100, or at least about 150, or between about 75 and about 500, or between about 100 and about 400, or between 100 and 250, copies of the extrachromosomal polynucleotide. In an embodiment, a high copy number plasmid useful for the invention has a 2micron origin of replication (known in the art as YEp plasmids). Examples of yeast high copy number plasmid include, but are not limited to, Gateway^{TM} pENTR (Invitrogen) and pRS420 (Christianson et al., 1992).

The yeast extrachromosomal polynucleotides, for example plasmids, described herein typically contain a yeast origin of replication, an antibiotic resistance gene, a bacterial origin of replication (for propagation in bacterial cells), multiple cloning sites, and a yeast nutritional gene for maintenance in yeast cells. The nutritional gene (or “auxotrophic marker”) is most often one of the following: 1) TRP1 (Phosphoribosylanthranilate isomerase); 2) URA3 (Orotidine-5′-phosphate decarboxylase); 3) LEU2 (3-Isopropylmalate dehydrogenase); 4) HISS (Imidazoleglycerolphosphate dehydratase or IGP dehydratase); or 5) LYS2 (α-aminoadipate-semialdehyde dehydrogenase). However, there are many known nutritional markers including those mentioned herein. Furthermore, the nutritional gene marker for a plasmid will be different to that for the reporter gene operably linked to the promoter activated by the yeast MAP kinase pathway.

Recombinant yeast of the invention comprises a reporter gene which either encodes a galactosidase or a selectable growth marker.

The “galactosidase” may be any enzyme which cleaves a terminal galactose residue(s) from a variety of substrates, and which is able to also cleave a substrate to produce a detectable signal. In an embodiment, the galactosidase is a β-galactosidase such as bacterial (for instance from E. coli) LacZ. In an alternate embodiment, the galactosidase is an α-galactosidase such as yeast (for instance S. cerevisiae) Mel-1 (Melcher et al., 2000; Aho et al., 1997). β-galactosidase activity may be detected using substrates for the enzyme such as X-gal (5-bromo-4-chloro-indolyl-β-D-galactopyranoside) which forms an intense blue product after cleavage, ONPG (o-nitrophenyl galactoside) which forms a water soluble yellow dye with an absorbance maximum at about 420 nm after cleavage, and CPRG (chlorophenol red-β-D-galactopyranoside) which yields a water-soluble red product measurable by spectrophotometry after cleavage. α-galactosidase activity may be detected using substrates for the enzyme such as o-nitrophenyl α-D-galactopyranoside which forms an indigo dye after cleavage, and chlorophenol red-α-D-galactopyranoside which yields a water-soluble red product measurable by spectrophotometry after cleavage. Kits for detecting galactosidase expression in yeast are commercially available, for instance the β-galactosidase (LacZ) expression kit from Thermo Scientific.

Preferably, the selectable growth marker is a nutritional marker or antibiotic resistance marker.

Typical yeast selectable nutritional markers include, but are not limited to, LEU2, TRP1, HIS3, HIS4, URA3, URA5, SFA1, ADE2, MET15, LYS5, LYS2, ILV2, FBA1, PSE1, PDI1 and PGK1. Those skilled in the art will appreciate that any gene whose chromosomal deletion or inactivation results in an unviable host, so called essential genes, can be used as a selective marker if a functional gene is provided on the, for example, plasmid, as demonstrated for PGK1 in a pgk1 yeast strain (Piper and Curran, 1990). Suitable essential genes can be found within the Stanford Genome Database (SGD) (http:://db.yeastgenome.org). Any essential gene product (e.g. PDI1, PSE1, PGK1 or FBA1) which, when deleted or inactivated, does not result in an auxotrophic (biosynthetic) requirement, can be used as a selectable marker on a, for example, plasmid in a yeast host cell that, in the absence of the plasmid, is unable to produce that gene product, to achieve increased plasmid stability without the disadvantage of requiring the cell to be cultured under specific selective conditions. By “auxotrophic (biosynthetic) requirement” we include a deficiency which can be complemented by additions or modifications to the growth medium.

Examples of antibiotic resistance genes (markers) include, but are not limited to, a chloramphenicol resistance gene, an ampicillin resistance gene, a tetracycline resistance gene, a Zeocin resistance gene, a spectinomycin resistance gene and a kanamycin resistance gene.

Yeast cells are typically transformed by chemical methods (e.g., as described by Rose et al., 1990, Methods in Yeast Genetics, Cold Spring Harbor Laboratory Press, Cold Spring Harbor, N.Y., and in Kawai et al., 2010). The cells are typically treated with lithium acetate to achieve transformation efficiencies of approximately 10⁴ colony-forming units (transformed cells)/μg of DNA. Other standard procedures for transforming yeast include i) the spheroplast method which, as the name suggests, relies on the production of yeast spheroplasts, ii) the biolistic method where DNA coated metal microprojectiles are shot into the cells, and iii) the glass bead methods which relies on the agitation of the yeast cells with glass beads and the DNA to be delivered to the cell. Of course, any suitable means of introducing nucleic acids into yeast cells can be used.

It is well known that transformation of organisms, such as yeast, with exogenous plasmids can lead to clonal differences in the penetrance of the transformed gene, due to differences in copy number or other factors. It is therefore advisable to screen two or more independent clonal isolates for each transformed receptor in order to maximise the likelihood of identifying suitable receptor=ligand pairs during screening. Different clonal isolates may be screened independently or may be combined into a single well for screening. The latter option may be particularly convenient where a nutritional reporter is used rather than a colorimetric reporter.

### Library Screening

The yeast of the invention can be used to test a small number of candidate compounds (ligands) for binding and activating a GPCR, or may be used to screen a library of compounds. The invention may use a “library” of test compounds, a library of different GPCRs, or a combination thereof.

As used herein, a “library” or “population” refers to a collection of many different compounds or yeast cells, for example, at least 10, at least 25, at least 50, at least 100, at least 500, at least 1,000, or at least 10,000, different compounds or yeast cells. Generally, a population may comprise each of the different yeast cells in a single container (for example culture), whereas a library generally has separate containers (for example wells on a microtitre plate) for each different yeast expressing a different GPCR. The compounds in the library may be related in structure, be completely different, or a mixture thereof. Examples of related compounds include a library of polypeptides which are at least 75%, or at least 90%, or at least 95%, identical to each other. As the skilled person would appreciate, some of the compounds in a library may be the same. In an embodiment, the library is a population of different yeast of the invention. In another embodiment, a library of potential ligands is used to screen against a yeast expressing a particular nematode GPCR. In a further embodiment, two or more potential ligands are screened against two or more different nematode GPCRs.

Screening may be performed by using one of the methods well known to the practitioner in the art, such as in microwells, by labelled cell sorting, or by growth on (or in) media (such as agar plates) where the reporter is a nutritional marker.

In a preferred embodiment, when measuring galactosidase activity the yeast are cultured in a microwell plate(s) comprising, for example, 48, 96, 384 or more wells.

In an alternate embodiment, when measuring expression of a selectable growth marker the yeast can be cultured on agar plates comprising (for example, an antibiotic) or lacking (for example, a nutrient to be compensated by expression of the reporter) the suitable reagent necessary to detect expression of the reporter. As the skilled person would appreciate, such selection could also be performed in liquid cultures and, for example, O.D. of the cells measured.

The assay conditions may be varied to take into account optimal binding conditions for different binding ligands of interest or other biological activities. Thus, the pH, temperature, salt concentration, volume and duration of binding, etc. may all be varied to achieve binding of ligand to the GPCR under conditions which resemble those of the environment of interest.

In one embodiment, the test compounds are small, organic non-peptidic compounds. In another embodiment, the test compound is a peptide or peptidomimetic. Exemplary methods for the synthesis of molecular libraries can be found in the art, for example in: Erb et al. (1994) and Gallop et al. (1994).

In an embodiment, the test compound is volatile. For the screening of a library of volatile compounds, the yeast can be exposed to air or other gas mixtures comprising the compound, or the compound can be exposed to a solution or suspension of the volatile compound in the yeast culture media (for example, the compound can be dissolved in the yeast culture media), if the compound is water soluble or water-immiscible respectively, or a suitable substrate may be soaked in the compound and placed over the yeast in culture, or any other suitable means can be used.

In some embodiments, a mixture of test compounds may be used or the test compound may be present in a complex mixture of compounds, only some of which are relevant to the purpose of the screen. Such mixtures potentially include expired human breath, saliva, urine, serum, plasma, whole blood, tears, sweat, faeces and a range of other biological specimens, as well as other types of mixtures such as beverages and wine.

In certain embodiments, the test compounds are exogenously added to the yeast cells expressing a recombinant receptor and compounds that modulate signal transduction via the receptor are selected. In other embodiments, the yeast cells also express the compounds to be tested.

In one embodiment, multiple independent clones of transformed yeast are tested for each receptor of interest. They may be screened as a single clone per well or multiple clones may be combined in a single well.

### Detection of Cyclohexanone

Cyclohexanone is a marker for some explosives, and hence it is desirable to have means for detecting this molecule.

As shown herein, both Str-144 (SEQ ID NO: 26) and Srj-22 (SEQ ID NO: 287) bind cyclohexanone. Thus, these polypeptides (receptors), or cyclohexanone binding variants thereof, can be used in methods for detecting cyclohexanone.

As the skilled person would appreciate, a wide variety of different detection systems can be used to detect a ligand binding a receptor protein.

In one embodiment, the method relies on detecting resonance energy transfer (RET) using, for example, bioluminescent resonance energy transfer (BRET) or fluorescence resonance energy transfer (FRET).

Light-emitting systems have been known and isolated from many luminescent organisms including bacteria, protozoa, coelenterates, molluscs, fish, millipedes, flies, fungi, worms, crustaceans, and beetles, particularly click beetles of genus Pyrophorus and the fireflies of the genera Photinus, Photuris, and Luciola. Additional organisms displaying bioluminescence are listed in WO 00/024878, WO 99/049019 and Viviani (2002).

One very well known example is the class of proteins known as luciferases which catalyze an energy-yielding chemical reaction in which a specific biochemical substance, a luciferin (a naturally occurring fluorophore), is oxidized by an enzyme having a luciferase activity (Hastings, 1996). A great diversity of organisms, both prokaryotic and eukaryotic, including species of bacteria, algae, fungi, insects, fish and other marine forms can emit light energy in this manner and each has specific luciferase activities and luciferins which are chemically distinct from those of other organisms. Luciferin/luciferase systems are very diverse in form, chemistry and function. Examples of bioluminescent proteins with luciferase activity may be found in U.S. Pat. Nos. 5,229,285, 5,219,737, 5,843,746, 5,196,524, and 5,670,356. Two of the most widely used luciferases are: (i) Renilla luciferase (from R. reniformis), a 35 kDa protein, which uses coelenterazine as a substrate and emits light at 480 nm (Lorenz et al., 1991); and (ii) Firefly luciferase (from Photinus pyralis), a 61 kDa protein, which uses luciferin as a substrate and emits light at 560 nm (de Wet et al., 1987).

Gaussia luciferase (from Gaussia princeps) has been used in biochemical assays (Verhaegen et al., 2002). Gaussia luciferase is a 20 kDa protein that oxidises coelenterazine in a rapid reaction resulting in a bright light emission at 470 nm.

Luciferases useful for the present invention have also been characterized from Anachnocampa sp (WO 2007/019634). These enzymes are about 59 kDa in size and are ATP-dependent luciferases that catalyze luminescence reactions with emission spectra within the blue portion of the spectrum.

Alternative, non-luciferase, bioluminescent proteins that can be employed in this invention are any enzymes which can act on suitable substrates to generate a luminescent signal. Specific examples of such enzymes are β-galactosidase, lactamase, horseradish peroxidase, alkaline phophatase, β-glucuronidase and β-glucosidase. Synthetic luminescent substrates for these enzymes are well known in the art and are commercially available from companies, such as Tropix Inc. (Bedford, Mass., USA).

An example of a peroxidase useful for the present invention is described by Hushpulian et al. (2007).

The choice of the substrate can impact on the wavelength and the intensity of the light generated by the bioluminescent protein. A widely known substrate is coelenterazine which occurs in cnidarians, copepods, chaetgnaths, ctenophores, decapod shrimps, mysid shrimps, radiolarians and some fish taxa (Greer and Szalay, 2002). For Renilla luciferase for example, coelenterazine analogues/derivatives are available that result in light emission between 418 and 512 nm (Inouye et al., 1997). A coelenterazine analogue/derivative (400A, DeepBlueC) has been described emitting light at 400 nm with Renilla luciferase (WO 01/46691). Other examples of coelenterazine analogues/derivatives are EnduRen and ViviRen.

As used herein, the term “luciferin” refers to a class of light-emitting biological pigments found in organisms capable of bioluminescence, which are oxidised in the presence of the enzyme luciferase to produce oxyluciferin and energy in the form of light. Luciferin, or 2-(6-hydroxybenzothiazol-2-yl)-2-thiazoline-4-carboxylic acid, was first isolated from the firefly Photinus pyralis. Since then, various forms of luciferin have been discovered and studied from various different organisms, mainly from the ocean, for example fish and squid, however, many have been identified in land dwelling organisms, for example, worms, beetles and various other insects (Day et al., 2004; Viviani, 2002).

There are a number of different acceptor molecules that can be employed in this invention. The acceptor molecules may be a protein or non-proteinaceous. Examples of acceptor molecules that are protein include, but are not limited to, green fluorescent protein (GFP), blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz, GFPuv, destabilised EGFP (dEGFP), destabilised ECFP (dECFP), destabilised EYFP (dEYFP), HcRed, t-HcRed, DsRed, DsRed2, t-dimer2, t-dimer2(12), mRFP1, pocilloporin, Renilla GFP, Monster GFP, paGFP, Kaede protein or a Phycobiliprotein, or a biologically active variant or fragment of any one thereof. Examples of acceptor molecules that are not proteins include, but are not limited to, Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, Texas Red, rare earth element chelates, or any combination or derivatives thereof.

One very well known example is the group of fluorophores that includes the green fluorescent protein from the jellyfish Aequorea victoria and numerous other variants (GFPs) arising from the application of molecular biology, for example mutagenesis and chimeric protein technologies (Tsien, 1998). GFPs are classified based on the distinctive component of their chromophores, each class having distinct excitation and emission wavelengths: class 1, wild-type mixture of neutral phenol and anionic phenolate:class 2, phenolate anion:class 3, neutral phenol:class 4, phenolate anion with stacked s-electron system:class 5, indole:class 6, imidazole:and class 7, phenyl.

A naturally occurring acceptor molecule which has been mutated (variants) can also be useful for the present invention. One example of an engineered system which is suitable for BRET is a Renilla luciferase and enhanced yellow mutant of GFP (EYFP) pairing which do not directly interact to a significant degree with one another alone in the absence of a mediating protein(s) (in this case, the G protein coupled receptor) (Xu et al., 1999).

In another embodiment, the acceptor molecule is a fluorescent nanocrystal. In an alternate embodiment, the acceptor molecule is a fluorescent microsphere.

In an embodiment, the receptor is labelled as generally described in WO 2010/085844. For instance, a) a bioluminescent protein is incorporated into the fifth non-transmembrane loop of the receptor, and an acceptor molecule incorporated into the C-terminus of the receptor, or b) the acceptor molecule forms part of the fifth non-transmembrane loop of the receptor, and the bioluminescent protein forms part of the C-terminus. Upon cyclohexanone binding the labelled receptor, in the presence of a substate for the bioluminescent protein, a modulation in BRET between the bioluminescent protein and the acceptor molecule occurs due to the spatial location and/or dipole orientation of the bioluminescent protein relative to the acceptor molecule being altered.

In a further embodiment of the method described in WO 2010/085844, the bioluminescent protein is a Renilla luciferase or a biologically active variant (such as RLuc2 or RLuc8) or fragment thereof, the acceptor molecule is green fluorescent protein 2 (GFP²), and the substrate is Coelenterazine 400a.

The labelled receptor may be present in a cell-free composition comprising the receptor embedded in a lipid bilayer, such as a liposome or a cell membrane preparation (for instance a yeast cell membrane preparation).

In a further embodiment, the method of detecting cyclohexanone of the invention is performed using microfluidics. For instance, the methods described in WO 2013/155553 can be used to detect cyclohexanone. More specifically, a sample which may comprise cyclohexanone is flowed through a microfluidic device comprising one or more microchannels along with a) the receptor labelled with a chemiluminescent donor domain and an acceptor domain, wherein the separation and relative orientation of the chemiluminescent donor domain and the acceptor domain, in the presence and/or the absence of cyclohexanone, is within ±50% of the Forster distance, and b) a substrate of the chemiluminescent donor. The labelled receptor molecule, sample and substrate are mixed in the device, wherein the spatial location and/or dipole orientation of the chemiluminescent donor domain relative to the acceptor domain is altered when the cyclohexanone binds the labelled polypeptide. Any modification of the substrate by the chemiluminescent donor is detected using an electro-optical sensing device.

Other examples of systems which can be used to detect resonance energy transfer (RET) as a result of cyclohexanone being a labelled polypeptide (receptor) as defined herein can be those generally described in WO 2004/057333.

Alternatively, the labelled receptor may be present in the cell membrane of an intact cell. Any cell expression system can be used, e. g., yeast, or mammalian (for example HEK293, CHO or COS cells) cell expression systems. Cells that normally express odorant receptors can be used. Isolation and/or culturing of such cells and their transformation with the olfactory receptor-expressing sequences of the invention can be done with routine methods (Vargas, 1999; Coon et al., 1989).

Several methods of measuring G-protein activity are known to those of skill in the art and can be used in conjunction with the methods of the present invention, including but not limited to measuring calcium ion or cyclic AMP concentration in the cells. Such methods are described in Howard et al. (2001), Krautwurst et al. (1999), Chandrashekar et al. (2000), Oda et al. (2000) and Kiely et al. (2007).

To evaluate electrophysiologic effects of cyclohexanone binding to cell-expressed receptor, patch-clamping of individual cells can be done. Patch-clamp recordings of the receptor cell membrane can measure membrane conductances. Some conductances are gated by odorants in the cilia and depolarize the cell through cAMP- or IP3-sensitive channels, depending on the species. Other conductances are activated by membrane depolarization and/or an increased intracellular Ca²⁺ concentration (Trotier, 1994).

Changes in calcium ion levels in the cell after exposure of the cell to cyclohexanone can be detected by a variety of means. For example, cells can be pre-loaded with reagents sensitive to calcium ion transients. Techniques for the measurement of calcium transients are known in the art. For example, Kashiwayanagi (1996) measured both of inositol 1,4,5-trisphosphate induces inward currents and Ca2+ uptake in frog olfactory receptor cells.

In certain specific embodiments, intracellular calcium concentration is measured by using a Fluorometric Imaging Plate Reader (“FLIPR”) system (Molecular Devices, Inc.). Other physiologic activity mechanisms can also be measured, e.g., plasma membrane homeostasis parameters (including lipid second messengers), and cellular pH changes (see, e.g., Silver, 1998).

Alternatively, in vitro synthesised mRNA coding for the receptor can be injected into Xenopus oocytes allowing electrophysiological or calcium imaging of cyclohexanone driven cell excitation.

In a further example, cyclohexanone is detected using a method relying on yeast cells, and methods of using such yeast cells, of the invention (see, for instance, Example 3).

### EXAMPLES

### Example 1

### Production of Nematode GPCR Library in Yeast

### Yeast Strain

The genotype of the parental strain of haploid yeast (Saccharomyces cerevisiae) that was used was: BY4741; MATa; his3Δ1; leu2Δ0; met15Δ0; ura3Δ0; ste2::kanMX4.

In order to make the screening system sensitive and robust, it is important to knock out the sst-2 gene to increase the sensitivity of the MAP kinase transduction cascade and the far-1 gene to avoid cell death due to overstimulation of this transduction pathway (FIGS. 1 to 3). The endogenous Ga (gpa-1) gene must also be deleted so that its protein does not compete with exogenous Ga subunits for access to Gβγ. ste-2, sst-2 and far-1 can be knocked out without any detrimental effect on the phenotype of the strain. However, gpa-1 is an essential gene that is required for yeast viability, as the absence of GPA1 protein would lead to free Gβγ, activating the transduction pathway and resulting in growth arrest at G1. Therefore, the inventors replaced the endogenous Gα (gpa-1) with a chimaeric Gβγ by replacing the last five amino acids of the yeast gpa-1 with the corresponding amino acids from a nematode olfactory Gα (odr-3, KAGGM) using homologous recombination.

The final strain was called Cyb-KAGMM.

### Selection of Appropriate Promoters for Reporters of Nematode Chemosensory G-Protein Coupled Receptor Activation

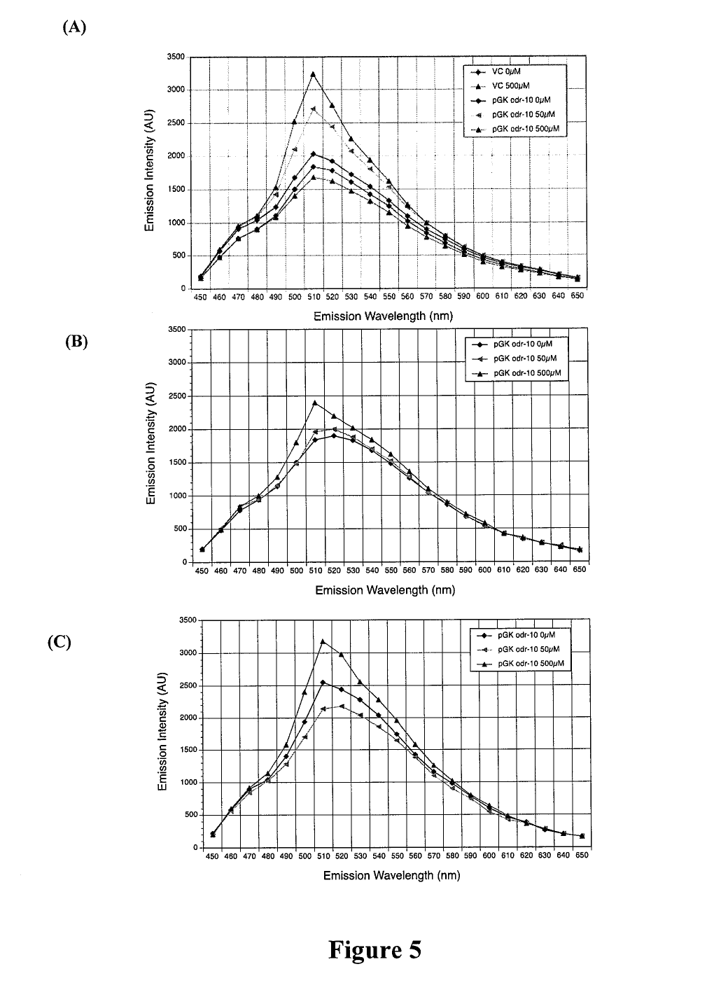

The FUS-1 promoter has commonly been used to drive expression of reporters in response to activation of the MAP kinase transduction pathway by heterologously expressed GPCRs. However, in the system the inventors have identified for nematode chemoreceptors, reporter expression was leaky (see, for example, FIGS. 5 to 7). This is particularly problematic for a screening assay because a high and/or varying background compromises the discriminating power of the assay. For example, leaky expression decreases the z-factor, which is a quantitative measure of the quality of the assay.

The inventors therefore tested the FIG-1 and FIG-2 promoters, other MAP-kinase dependent promoters, which significantly reduced background leaky expression of the reporter gene as shown in FIGS. 7a and 7b. This was a surprising advantage of the FIG-1 and FIG-2 promoters that the inventors had not been led to expect from their knowledge of the prior art.

### Selection of Promoter for Expression of Nematode Chemosensory G-Protein Coupled Receptor

The inventors investigated suitable promoters for reliably expressing nematode chemosensory GPCRs. The inventors picked two strong promoters: the galactose-inducible GAL-1 promoter, as used by Minic et al. (2005) for expressing the rat 17 olfactory receptor and the constitutive PGK-1 promoter used by Dowell's group (Dowell and Brown, 2002 and 2009) for non-chemosensory mammalian GPCRs. Glucose was used as carbon source for the system using the PGK-1 promoter, whereas a raffinose/galactose mixture was used to induce the GAL-1 promoter, following standard protocols.

Surprisingly in the light of the nearest prior art (Minic et al, 2005), using the GAL-1 promoter the inventors were unable to observe reproducible ODR-10 expression by western blotting nor could they reproducibly generate a functional receptor response using this promoter (results not shown), but when expressed under the control of the constitutive PGK-1 promoter the inventors were able to observe consistent expression of a GFP² labelled variant of ODR-10 using laser confocal microscopy (FIG. 4). The inventors therefore proceeded to test the system with positive and negative control receptors and both a known ligand (diacetyl) and a known non-ligand (acetoin).

### Cloning of Nematode Chemosensory G-Protein Coupled Receptors

Receptor cDNAs were inserted into a Gateway® pENTR vector and thence into a modified Gateway® pDEST-His plasmid under the control of the PGK-1 promoter as mentioned above. To confirm proper functioning of this system it was tested with odr-10 cDNA in the Cyb-KAGMM yeast strain. ODR-10 is a nematode chemoreceptor GPCR that responds selectively and with high affinity to butane-2,3-dione (diacetyl). The H110Y variant of ODR-10 bears a single point mutation that ablates diacetyl 30 responsiveness. It is therefore an excellent negative control and surrogate for other non-responding GPCRs. The inventors cloned both wild type and H110Y mutant variants of odr-10 cDNA into the Gateway® cassette downstream of the PGK-1 promoter. GFP and LacZ reporters were cloned under either the FIG-2 or FUS-1 promoters in a pESC-Leu plasmid. The gpa-1/odr-3 chimaera, which contains the last five amino acids of nematode ODR-3, was chromosomally integrated at the gpa-1 locus.

The precise methodology was as follows:

### Growth

A single colony was picked and inoculated into 10 mL of standard yeast drop-out selection medium (SD) without leucine and histidine and supplemented with 2% (w/v) glucose. This was incubated for 16 hrs at 30° C. with 180 rpm shaking in a 50 mL Falcon tube.

### Assessment of Alternative Reporters

### i) GFP²

GFP has been used successfully as a reporter for some mammalian GPCRs (Fukutani et al., 2012). If feasible it has the advantage of providing a simple readout with no need for workup after induction/expression.

The cells were adjusted to give an ABS_{600 nm}≈1. Diacetyl was added at a concentration in the range of 1-1000 μM in 1 mL of medium in a 2 mL or larger tube, and shaken for 7 hrs at 30° C. at 130 rpm before being scanned for fluorescence emission between 450 and 650 nm with 420 excitation wavelength for GFP. However, GFP expression was very variable between biological repeats of the same experiment as shown in FIG. 5. Furthermore, even in the best case (FIG. 5a) there was less than a twofold difference between the peak GFP responses to 500 μM diacetyl with ODR-10 expression and for the vector control. The combination of variability and narrow dynamic range of the responses, even in this ideal situation with a known ligand and receptor pair, would make practical use of such an assay for ligand discovery with a variety of receptors, impractical or impossible.

### ii) Lac-Z

The inventors also tested the suitability of the Lac-Z marker, which encodes β-galactosidase and therefore requires an enzymatic development step after induction/expression. The inventors repeated the same growth and induction procedure but after the 7 hrs induction, one of the following protocols was performed:

a) X-Gal Workup

Colour was developed using the HTX Kit (Cat #P01002) from Dualsystems Biotech according to their instructions.

b) ONPG Workup
The cells were centrifuged and the supernatant was discarded.
100 μl of 100 mM sodium phosphate buffer, pH 7.5, containing 0.1% sodium dodecyl sulphate was added to the tubes
The tubes were shaken at 200 rpm for 10 min at room temperature.
One quarter volume of 2.5 mM ortho-nitrophenyl-βD-galactopyranoside (ONPG) was added at room temperature to give a final ONPG concentration of 0.5 mM.
The reaction was stopped by adding 1M Na₂CO₃ to a final concentration of 0.3M.
Absorbance was measured at 414 nm.

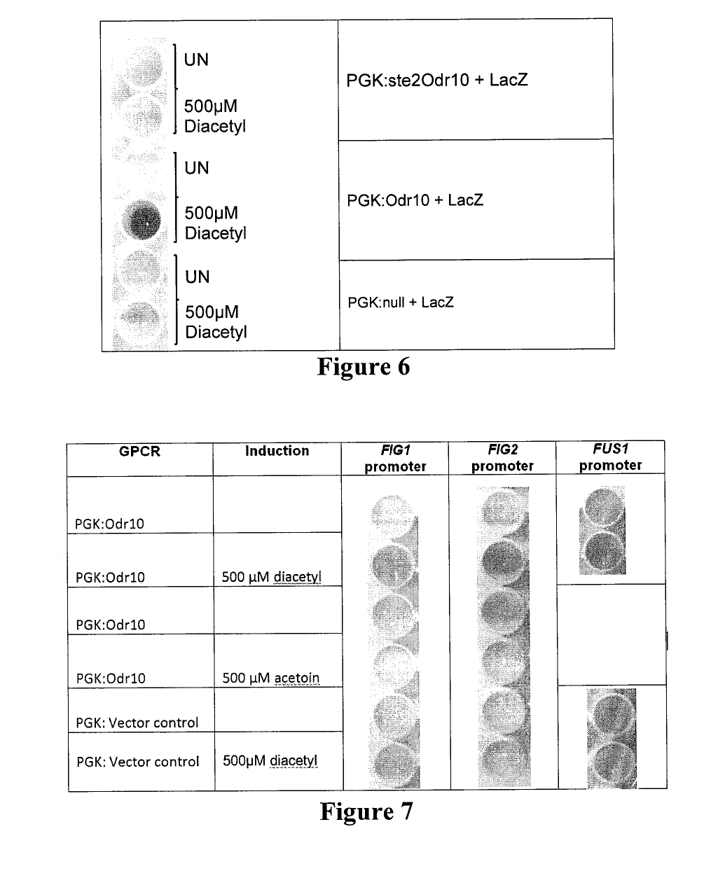

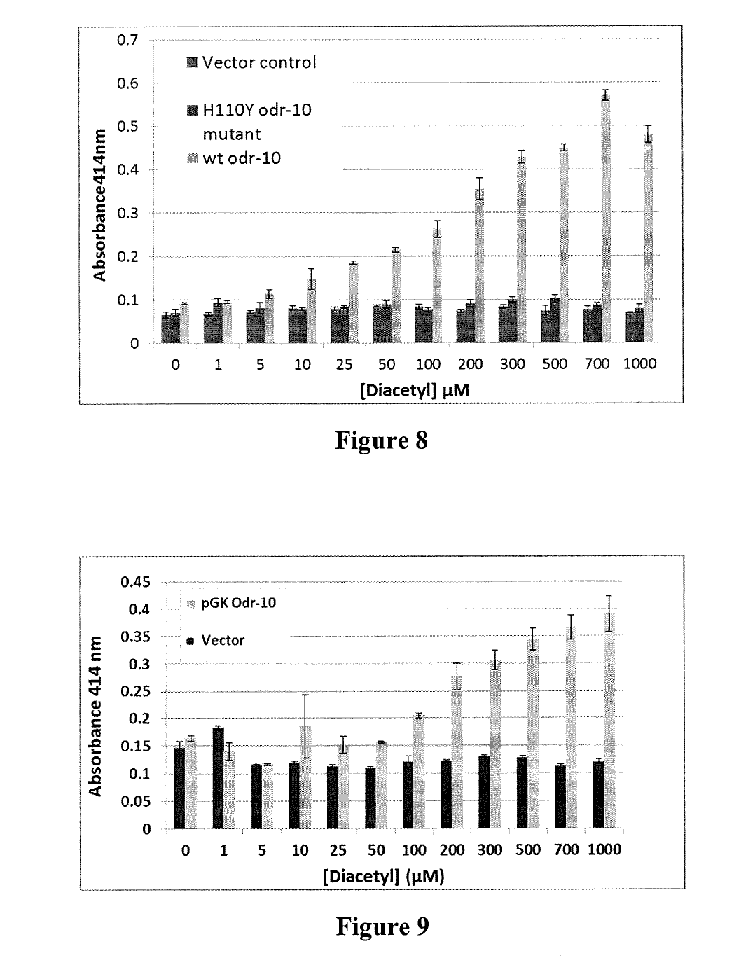

The assays performed with LacZ under the control of either FUS-1 or FIG-2 promoters both gave strong signals that were easily distinguishable by eye from controls (FIGS. 6 and 7) and that were reproducible across biological repeats carried out on separate days (results not shown). Both X-gal (FIG. 6) and ONPG (FIG. 7) substrates gave measurable signals. However, ONPG is a more cost effective substrate and quantitative experiments were therefore carried out with that substrate. The maximum increase in signal, observed with 700 μM diacetyl when driven by FUS-2, was approximately 6 fold higher than background. The background was very stable (FIG. 8). Under the FUS-1 promoter the maximum ligand-induced increase in signal occurred at 1 mM and was only a factor of approximately 2.5 fold (FIG. 9). Furthermore, the background was higher and more variable.

### Nematode Chemoreceptor Gene Library for Screening

A large library of C. elegans receptors that can be screened for responses to explosive targets has been constructed. Five putative chemoreceptor subfamilies containing a putative 578 receptors were selected as source material for the library (FIG. 10).

The sequences of these genes were accessed from the online database for C. elegans: Wormbase. In order to clone each individual gene, total RNA from a mixed life stage culture of C. elegans was prepared using commercially available RNA extraction kits. cDNA was prepared using Invitrogen's superscript III reverse transcriptase. Primers for the amplification of each individual gene were designed to include the ATG start codon (forward primer) and the STOP codon (reverse primer) to ensure the full gene is amplified. A proof reading polymerase was used in standard PCR reactions to amplify the genes.

Amplification products of appropriate size were cloned into pENTR/D-TOPO vector using Invitrogen's Gateway® cloning system. The Gateway® system is highly efficient and circumvents limitations of traditional restriction enzyme cloning as well as enabling simple sub-cloning into a multitude of vectors for different downstream scientific studies.

The inventors were surprised to find that transcription of C. elegans putative chemoreceptor GPCRs is quite promiscuous, with many splice variants of genes present in cDNA samples. In order to ensure the correct sequence is cloned, multiple colonies were picked for each gene and their correct insert size was checked by restriction digestion. The sequences of correctly sized clones were compared back to the available sequence on Wormbase and error free clones were selected for inclusion in the C. elegans chemoreceptor library.

Sequences and detailed information for each clone were recorded electronically and clones were stored at −20° C. A total of 297 error free receptor clones were isolated from the pool of 578, which represents the majority of expressed, accessible and useful sequences within the str subfamily.

The sub-cloning of the receptors from the pENTR D Topo vector into a yeast expression vector was completed. The expression vector was designated pDEST PGK-HIS. It is based on the yeast expression vector pESC-HIS (Stratagene) wherein the GAL1 and GAL10 promoters were replaced with a single (700 bp) yeast PGK promoter, and a Gateway® attR1-attR2 cassette was inserted immediately downstream of the PGK promoter. Transfer was accomplished using a standard Gateway® LR Clonase reaction according to InVitrogen's instructions.

Duplicate independent clones of yeast transformed with 294 of the receptors were selected onto 8×96 position master plates (up to 37 receptors, in duplicate, per plate with room for positive and negative controls). Seven of eight master plates have been transferred to 96 well screening plates, along with positive and negative controls and screened against at least one chemical target.

### Example 2

### Screening of Nematode GPCR Library in Yeast

In order to be viable for screening multiple receptors against a single chemical or a mixture of chemicals simultaneously, or a single receptor against multiple chemicals or mixtures, it is convenient and efficient to perform the assay in a multiwell plate format, for example 48, 96, 384 or other higher density formats. As proof of principle, the inventors performed a control assay in 48 and 96 well formats.

### Method

The method was as follows (adpated from Brouchon-Macari et al., 2003):
1 mL of Saccharomyces cerevisiae Minimal Medium (SCMM)-His, -Leu was inoculated with a single colony of the screening strain transformed with odr-10 (lacZ/Odr10/gpa1-KAGMM/ste2⁻sst2⁻far1³¹ gpa1⁻) at Abs_{600 nm}=1, in a 96-well deep well plate.
The plate was incubated overnight (18 hours) at 30° C. with shaking at 900 rpm.
The culture from the well was diluted into fresh wells (96 or 48 well plate) to a final volume of 1 mL at the following yeast cell optical densities:
   1. Abs₆₀₀=1
   2. Abs₆₀₀=0.5
   3. Abs₆₀₀=0.5
Wells 1 and 2 above were induced with 500 μM of a diacetyl solution (made up fresh and kept on ice); 1M: 87 μL diacetyl+913 μL water; then diluted 1/10 in water and 5 μL added to 1 mL cells for a final concentration of 500 μM). All wells were incubated for 30° C. for 18 hours with shaking at 900 rpm.
The plate was centrifuged to pellet the cells, the supernatant was aspirated off and discarded and the pellet was resuspended in 120 μL of lysis buffer (100 mM sodium phosphate buffer, pH 7.5 (consisting of 82 mM Na₂HPO₄ and 12 mM NaH₂PO₄ with 0.1% (w/v) sodium dodecyl sulfate). Initial absorbance was read at 414 nm in a plate-reading spectrophotometer.
The plate was shaken at 900 rpm for 10 min at 30° C.
Thirty microlitres of 2.5 mM o-nitrophenyl-β-D-galactopyranoside (ONPG) (10 mg ONPG dissolved in 130 μL dimethyl formamide is 250 mM, then dilute 1/100 in water for final concentration, which is 2.5 mM) was added and the plate was incubated at room temperature for between 5 minutes and 3 hours.
Eighty microlitres of a 1M Na₂CO₃ solution was added and the absorbance was read in a plate-based spectrophotometer, either at 414 nm or a spectrum was recorded for each well over the visible region.

### Results

In some cases, colour development was visible by eye as early as five minutes after adding substrate. Abs₄₁₄ readings after 3 hours were: 0.247 for the Abs_{600 nm}=1 starting condition+diacetyl, 0.521 for the Abs_{600 nm}=0.5 starting condition+diacetyl and 0.165 for the Abs_{600 nm}=0.5 starting condition with no added diacetyl.

The assay was repeated with a range of different dilutions (¼ to 1/20) of the starting yeast culture in the wells of either 96 or 48 deep-well plates (the former can contain a total of 2 mL and the latter 5 mL.) Normally dilutions required to reach Abs_{600 nm}=1 are in the range of ½ to ¼. This experiment probed a set of higher dilutions. Incubation was for 17 hours at 30° C. and 900 rpm shaking. Diacetyl induction of lacZ was detected in all the conditions except the 1/20 cell dilution in the 48 well plate. 96 well plates consistently gave higher diacetyl-induced to control Abs_{414 nm} ratios at all dilutions than 48 well plates. In 96 well plates, the higher cell dilutions consistently gave the better diacetyl-induced to control ratios (FIG. 11). The best ratio was in the case of the 96-well plate and the 1/20 dilution where the diacetyl-induced absorbance was 0.481 and the uninduced control was 0.079.

### Conclusion

The present inventors tested a large number of published procedures and were surprised to find none were useful for producing yeast where ligand induced binding to heterologously expressed nematode GPCRs produced a suitably reliable and sensitive signal through a reporter. Examples of previously published procedures that did not work included that described by Minic et al. (2005) with a chimeric Gα, Gal inducible promoters for the receptors, nematode Gα replacing yeast Gα, and having the nematode GPCR cassette and the reporter construct on the same plasmid, whereas the use of GFP as a reporter was very unreliable and the commonly used FUS-1 as a promoter for the reporter resulted in a weak signal and higher background. As a result, a new assay had to be developed to meet the inventor's requirements as to reliability and sensitivity. In this regard, initial experiments have shown a z factor of 0.94 which is excellent and surprising.

### Example 3

### Screening of Nematode GPCR Library in Yeast to Cyclohexanone

In order to screen multiple nematode GPCRs against cyclohexanone simultaneously, a high throughput 96-well plate assay was designed.

### Method

The method was adapted from (Brouchon-Macari et al., 2003).

500 μL aliquots of Saccharomyces cerevisiae Minimal Medium (SCMM)-Urea,-His,-Leu (SCMM-UHL) containing 2% glucose, were inoculated with single colonies of the screening strain transformed with selected nematode GPCRs (pFIG2:LacZ/pPGK:GPCRx/gpal-KAGMM/ste2-sst2- far1- gpa1-) in a 96-well deep well plate. Colonies H9, H10 were positive controls transformed with odr-10, which responds to diacetyl; H11 and H12 were negative controls transformed with mutated odr-10, which does not respond to diacetyl.

The plate was incubated overnight (24 hours) at 30° C. with shaking at 1000 rpm.

Each culture was diluted 1:10 into fresh wells of a 96-well plate to a final volume of 1 mL.

All wells, except control wells H9-12, were induced with 5 mM cyclohexanone (51.77 μL cyclohexanone was added into 100 mL SCMM-U-H-L medium containing 2% glucose). As control, H9-12 was induced with 500 μM diacetyl (1 M diacetyl was prepared fresh, 8.7 μL diacetyl+91.3 μL water, then dilute 1:100 in water and add 50 μL to 1 mL cells for 500 μM final). All wells were incubated at 30° C. for 18 hours with shaking at 1000 rpm.

The plate was centrifuged (1500×g, 5 min), the supernatant was discarded and the pellet was resuspended in 120 μL of lysis buffer [100 mM sodium phosphate buffer, pH 7.5 (consisting of 82 mM Na2HPO4 and 12 mM NaH2PO4) containing 0.1% (w/v) sodium dodecylsulfate (SDS)]. The plate was shaken at 1000 rpm for 10 min at 30° C.

40 μL of 2.5 mM o-nitrophenyl-β-D-galactopyranoside (ONPG) was added and the plate was incubated at room temperature for 10 min. 80 μL 1 M Na2CO3 solution was added to stop the reaction. The plate was centrifuged (1500×g, 5 min) and 100 μL of supernatant was carefully pipetted off each well to the wells of a fresh transparent 96-well plate. The key to the different well is provided in Table 2.

Results were recorded photographically and the spectrum of every well was recorded with a plate-reading spectrophotometer over the 380 nm to 500 nm region of the visible light spectrum.

### Results

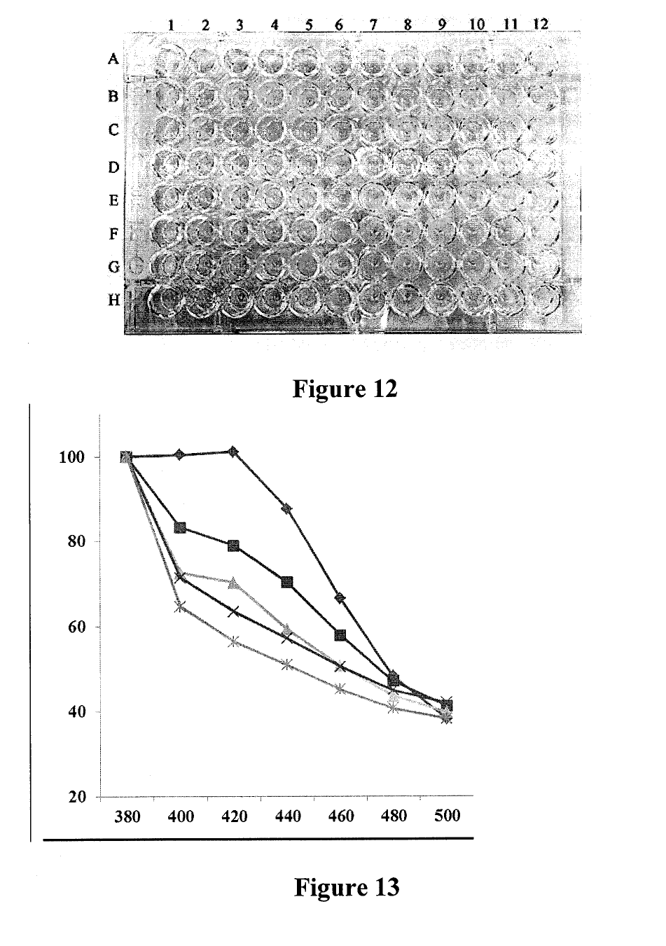

Yellow colour was observed in wells B7, D2 and H9 (FIG. 12), indicating they were induced by the tested ligands. Spectra reading from 380 nm to 500 nm were measured and analyzed (FIG. 13). The spectra of B7, D2 and H9 showed clear peaks at 420 nm, which were not observed from other wells.

As expected, positive control H9 (odr-10) responds to diacetyl, therefore showed yellow color and the spectra peak at 420 nm. Unlike H9, the other positive control H10 did not show any response (color or spectra) to diacetyl in this assay. This reflects the inventors' regular observation that different clonal yeast lines, transformed with the same receptor, can show different responses to an inducing ligand. It also explains why the inventors chose to screen two independent clones for all of the receptors in our library. As mentioned elsewhere herein, there would be advantages in screening a larger number of clones for each receptor if this could be managed logistically.

TABLE 2
Example of receptor assignments for plate 3 of the nematode GPCR
screenable library. Wells F7-12, G7-12, H7 and H8 are empty to avoid the control
wells (H9-12) being contaminated by the ligand used in the other wells and vice versa.

| | 1 | 2 | 3 | 4 | 5 | 6 | 7 | 8 | 9 | 10 | 11 | 12 |
|---|---|---|---|---|---|---|---|---|---|---|---|---|
| A | Odr10 | Odr10 | Odr10 | Odr10 | Str130 | Str130 | Str131 | Str131 | Str134 | Str134 | Str135 | Str135 |
| | | | mut | mut | | | | | | | | |
| B | Str139 | Str139 | Str141 | Str141 | Str143 | Str143 | Str144 | Str144 | Str146 | Str146 | Str148 | Str148 |
| C | Srj8 | Srj8 | Srj9 | Srj9 | Srj11 | Srj11 | Srj14 | Srj14 | Srj15 | Srj15 | Srj19 | Srj19 |
| D | Srj22 | Sr122 | Sri26 | Srj26 | Srj27 | Srj27 | Srj37 | Srj37 | Srd1 | Srd1 | Srd3 | Srd3 |
| E | Srd5 | Srd5 | Srd9 | Srd9 | Srd10 | Srd10 | Srd11 | Srd11 | Srh15 | Srh15 | Srh17 | Srh17 |
| F | Srh149 | Srh149 | Srh159 | Srh159 | Srh166 | Srh166 | | | | | | |
| G | Srh167 | Srh167 | Sri51 | Sri51 | Sri54 | Sri54 | | | | | | |
| H | Sri57 | Sri57 | Sri60 | Sri60 | Sri63 | Sri63 | | | Odr10 | Odr10 | Odr10 | Odr10 |
| | | | | | | | | | | | mut | mut |

“Odr-10 mut” is the odr-10 non-functional mutant H110Y.

As expected, negative control H11 and H12 (the mutated odr-10) did not show any response to 500 μM diacetyl (FIG. 13). Well G5 is an example of a well that did not show any peak in the spectra either (FIG. 13). Conversely, well B7 and D2 showed clear responses to cyclohexanone. According to Table 2, well B7 contains the yeast strain transformed with nematode GPCR Str144 and D2 is the yeast strain transformed with GPCR Srj22. This assay was repeated at least three times and the results were found to be consistent.

### Conclusion

In this example, the inventors did not only identify the candidate GPCRs for sensing cyclohexanone, but also successfully demonstrated a high throughput assay to screen multiple nematode GPCRs in yeast to the interest chemical compounds. Beside eye observation of the color change, the inventors also developed a reliable spectra analysis method to analyze this assay results and help identify the candidate receptors for the interested ligands.

### Example 4

### Other Markers

The inventors also envisage an assay of the type described above, wherein the reporter gene, rather than being lacZ or another visible marker, is a selectable growth marker such as a nutritional marker or antibiotic resistance marker, possibly selected from LEU2, TRP1, HIS3, HIS4, URA3, URA5, SFA1, ADE2, MET15, LYS5, LYS2, ILV2, FBA1, PSE1, PDI1 and PGK1, that is required for yeast growth in the screening buffer. In this case multiple independent clones of a single receptor sequence would be placed into each of the screening wells. On induction, only those clones capable of a positive response would grow and the extent of any growth would be an indication of a ligand-receptor “hit”. Such an arrangement would have the additional benefits, over the other examples shown here, of allowing greater clonal diversity to be screened simultaneously and also permitting the screening of up to at least 74 different receptor types per 96 well plate rather than the 37 demonstrated in Example 3.

The present application claims priority from AU2013901329 filed 16 Apr. 2013, and AU 2013901925 filed 30 May 2013, the entire contents of both of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Aho et al. (1997) Anal. Biochem. 253:270-272.
Brouchon-Macari et al. (2003) BioTechniques 35:446-448.
Brown et al. (2000) Yeast 16:11-22.
Chandrashekar et al. (2000) Cell 100: 703-711.
Christianson et al. (1992) Gene 110:119-122.
Coon et al. (1989) Proc. Natl. Acad. Sci. USA 86:1703-1707
Cuppen et al. (2003) Comp Funct Genomics 4:479-491.
Day et al. (2004) Luminescence 19:8-20.
de Wet et al. (1987) Mol. Cell. Biol. 2987:725-737.
Dowell and Brown (2002) Receptors and Channels 8:343-352.
Dowell and Brown (2009) G-Protein Coupled Receptors in Drug Discovery: Methods in Molecular Biology 552:213-229.
Erb et al. (1994) Proc. Natl. Acad. Sci. USA 91:11422.
Fredriksson and Schioth (2005) Molecular Pharmacology 67:1414-1425.
Frishman and Argos (1997) Proteins 27:329-335.
Fukutani (2012) Biotechnology and Bioengineering 109:205-212.
Gallop et al. (1994) J. Med. Chem. 37:1233.
Greer and Szalay (2002) Luminescence 17:43-74.
Hastings (1996) Gene 173:5-11.
Harayama (1998) Trends Biotechnol. 16:76-82.
Hofmann and Stoffel (1993) Biol. Chem. 374:166.
Hofmann et al. (2009) Trends Biochem Sci 34:540-552.
Howard et al. (2001) Trends Pharmacol Sci. 22:132-140.
Hushpulian et al. (2007) Biotransformation 25:2-4
Iguchi et al (2010) J. Biochem. 147:875-884.
Inouye et al (1997) Biochem. J. 233:349-353.
Jansen et al. (1999) Nat. Genet. 21:414-419.
Kashiwayanagi (1996) Biochem. Biophys. Res. Commun. 225:666-671.
Kawai et al. (2010) Bioeng. Bugs 1:395-406.
Kiely et al. (2007) J Neurosci Methods 159:189-194
Klein et al. (1984) Biochim. Biophys. Acta 787:221-226.
Krautwurst et al. (1999) Cell 95:917-926
Lorenz et al (1991) Proc. Natl. Acad. Sci. USA 88:4438-4442.
Melcher et al. (2000) Gene 247:53-61.
Minic et al. (2005) FEBS Journal 272:524-537.
Needleman and Wunsch (1970) J. Mol Biol. 45:443-453.
Oda et al. (2000) J Biol Chem. 275:36781-36786
Oldham abd Hamm (2008) Nature 9:60-71.
Persson and Argos (1994) J. Mol. Biol. 237:182-192.
Piper and Curran (1990) Curr. Genet. 17:119-123.
Robertson (1998) Genome Research 8:449-463.
Robertson (2001) Chem. Senses. 26:151-159.
Trotier (1994) Semin. Cell Biol. 5:47-54
Tsien (1998) Ann. Rev. Biochem. 63:509-544.
Vargas (1999) Chem. Senses 24:211-216
Verhaegen et al. (2002) Anal. Chem. 74:4378-4385
Viviani (2002) Cell. Mol. Life Sci. 59:1833-1850.
von Heijne (1992) J.Mol.Biol. 225:487-494.
Xu et al. (1999) Proc. Natl. Acad. Sci. USA. 96:151-156.
Zhang et al. (1999). Journal of Biomolecular Screening 4:67-73.

1. A yeast cell comprising
i) a first high copy number extrachromosomal polynucleotide comprising a first polynucleotide encoding a nematode G protein coupled receptor (GPCR) operably linked to a constitutive promoter,
ii) a second high copy number extrachromosomal polynucleotide comprising a second polynucleotide encoding a galactosidase, or a selectable growth marker, operably linked to a promoter activated by the yeast MAP kinase pathway, wherein the promoter is not a FUS-1 promoter,
iii) a mutated yeast gpa-1 gene, and
iv) a third polynucleotide encoding a chimeric G protein comprising the N-terminus of a yeast gpa-1 and at least four C-terminal amino acids of a nematode G protein, operably linked to a promoter,
wherein each promoter directs expression of the polynucleotides in the cell.

2. The yeast cell of claim 1, wherein the yeast further has a mutated sst-1 gene and a mutated far-1 gene.

3. The yeast cell of claim 1 or claim 2, wherein the yeast further has a mutated ste-2 gene.

4. The yeast cell according to any one of claims 1 to 3, wherein the promoter activated by the yeast MAP kinase pathway is the FIG-1 or FIG-2 promoter.

5. The yeast cell according to any one of claims 1 to 4, wherein the constitutive promoter operably linked to the first polynucleotide is selected from the PGK promoter, the ADH-1 promoter, ENO promoter, the PYK-1 promoter, and the CYC-1 promoter.

5. The yeast cell according to any one of claims 1 to 5, wherein the cell comprises at least 100 copies of each of the first and second high copy number extrachromosomal polynucleotides.

6. The yeast cell according to any one of claims 1 to 5, wherein the yeast is Saccharomyces cerevisiae.

7. The yeast cell according to any one of claims 1 to 6, wherein the yeast is haploid.

8. The yeast cell according to any one of claims 1 to 7, wherein the yeast is mating type a.

9. The yeast cell according to any one of claims 1 to 8, wherein the third polynucleotide is stably integrated into the genome of the yeast cell.

10. The yeast cell according to any one of claims 1 to 9, wherein the nematode is Caenorhabditis elegans.

11. The yeast cell according to any one of claims 1 to 10, wherein the nematode GPCR is a chemoreceptor, odorant receptor or taste receptor.

12. The yeast cell according to any one of claims 1 to 11, wherein the nematode GPCR comprises an amino acid sequence which is at least 80% identical to one or more of SEQ ID NO's 1 to 297.

13. The yeast cell according to any one of claims 1 to 12, wherein the chimeric G protein comprises about 5 C-terminal amino acids of the nematode G protein.

14. The yeast cell according to any one of claims 1 to 13, wherein the selectable growth marker is a nutritional marker or antibiotic resistance marker.

15. The yeast cell of claim 14, wherein the nutritional marker is selected from HIS3, ADE2, URA2, LYS2, ARG2, LEU2, TRP1, MET15, HIS4, URA3, URA5, SFA1, LYS5, ILV2, FBA1, PSE1, PDI1 and PGK1.

16. The yeast cell according to any one of claims 1 to 15, wherein the galactosidase is a β-galactosidase or an α-galactosidase.

17. The yeast cell of claim 16, wherein the β-galactosidase is LacZ.

18. The yeast cell according to any one of claims 1 to 17, wherein the promoter operably linked to the third polynucleotide encoding the chimeric G protein is the endogenous gpa-1 promoter.

19. The yeast cell according to any one of claims 1 to 18, wherein the first and second extrachromosomal polynucleotides are a plasmid.

20. A population or library of yeast cells according to any one of claims 1 to 19, wherein at least 10 of the yeast cells have different nematode GPCRs.

21. The population or library of claim 20, wherein on average each yeast cell comprises at least 100 copies of each of the first and second high copy number extrachromosomal polynucleotides.

22. The population or library of claim 20 or claim 21 which comprises at least 10, at least 25, at least 50, at least 100, at least 200, at least 250, or all of the nematode GPCRs which comprise an amino acid sequence provided as SEQ ID NO's 1 to 297.

23. A composition comprising a yeast cell according to any one of claims 1 to 19, or the population of yeast cells according to any one of claims 20 to 22.

24. A method of screening for a ligand that binds and activates a nematode G protein coupled receptor, the method comprising
i) contacting a yeast cell according to any one of claims 1 to 19 with a candidate ligand, and
ii) determining if the cell expresses galactosidase, or the selectable growth marker,
wherein the presence of galactosidase activity, or activity of the selectable growth marker, indicates that the ligand binds and activates a nematode G protein coupled receptor.

25. The method of claim 24, wherein the z factor of the method when the ligand binds and activates a nematode G protein coupled receptor is about 0.5 to 1.

26. The method of claim 24 or claim 25, wherein the ligand is volatile.

27. The method according to any one of claims 24 to 26, wherein the ligand is from a library of compounds.

28. The method according to any one of claims 24 to 27, wherein the method comprises screening a population or library according to any one of claims 20 to 23.

29. A method of detecting a ligand in a sample, the method comprising
i) contacting at least one yeast cell according to any one of claims 1 to 19 which comprises a nematode G protein coupled receptor which binds and is activated by the ligand,
ii) determining if the cell expresses galactosidase, or the selectable growth marker,
wherein the presence of galactosidase activity, or activity of the selectable growth marker, indicates that the ligand is present in the sample.

30. A method of detecting cyclohexanone in a sample, the method comprising
i) contacting the sample with a polypeptide which comprises an animo acid sequence provided as SEQ ID NO:26 or SEQ ID NO:287, or a variant thereof which binds cyclohexanone, and
ii) detecting whether the polypeptide is bound to cyclohexanone.

31. The method of claim 30, wherein the polypeptide is detectably labelled.

32. The method of claim 30 or claim 31, wherein the variant comprises an animo acid sequence which is at least 80% identical to SEQ ID NO:26 or SEQ ID NO:287, or a cyclohexanone binding fragment thereof.
